(19) United States
(12) Patent Application Publication
(10) Pub. No.: US 2026/0297541 A1
(43) Pub. Date: Oct. 1, 2026

(54) BACTERIOPHAGE VB_SBRT-PBOVINEB21 HAVING LYTIC ACTIVITY AND BIOFILM CONTROL ACTIVITY AGAINST STREPTOCOCCUS BOVIS/STREPTOCOCCUS EQUINUS COMPLEX AND LACTIC ACID-PRODUCING BACTERIA

(71) Applicant: GACHON UNIVERSITY OF INDUSTRY-ACADEMIC COOPERATION FOUNDATION, Seongnam-si, Gyeonggi-do (KR)

(72) Inventors: Ji Hyung KIM, Seongnam-si, Gyeonggi-do (KR); Seon Young PARK, Seongnam-si, Gyeonggi-do (KR); Hyemin KWON, Seongnam-si, Gyeonggi-do (KR)

(21) Appl. No.: 19/478,240

(22) PCT Filed: Apr. 8, 2024
(86) PCT No.: PCT/KR2024/004615

§ 371 Date: Oct. 23, 2025

(30) Foreign Application Priority Data
Apr. 27, 2023 (KR) 10-2023-0055197

(51) Int. Cl. C12N 7/00; A61K 35/76; A61P 31/04

(52) U.S. Cl. CPC C12N 7/00; A61K 35/76; A61P 31/04; C12N 2795/00031; C12N 2795/00032

(57) ABSTRACT

An embodiment relates to a bacteriophage vB_SbRt-pBovineB21 having excellent lytic activity and biofilm control activity against Streptococcus bovis/Streptococcus equinus complex (SBSEC) and lactic acid-producing bacteria, and the phage and/or composition according to the present invention can be used as an antibacterial agent, a disinfectant, and a feed additive for ruminants.

### TECHNICAL FIELD

The present invention was made under the support of the Ministry of Oceans and Fisheries, with project unique number 1525014590 and project number 202201286, wherein the research management specialized institution of the above project is the Korea Institute of Marine Science & Technology Promotion, the research project and research task name is “Technology development for bio-materialization of marine and fishery by-products,” the leading institution is the Korea Institute of Ocean Science & Technology, and the research period is from Jan. 1, 2023 to Dec. 31, 2023.

The present invention relates to a bacteriophage vB_SbRt-pBovineB21 having lytic activity and biofilm control activity against Streptococcus bovis/Streptococcus equinus complex (SBSEC) and lactic acid-producing bacteria, and more specifically, to a bacteriophage having bactericidal activity and biofilm control ability against bacteria that cause rumen acidosis in ruminants.

### BACKGROUND ART

Rumen acidosis is a dyspeptic disease that occurs due to excessive production of lactic acid in the rumen caused by a sudden change of feed or excessive feeding of concentrated feed during the management of ruminants. The cause of overproduction of lactic acid is that, when excessive feeding of compound feed or shortage of roughage, or selective eating of feed occurs, a large intake of feed containing a large amount of starch leads to a rapid increase in the number of starch-degrading bacteria, and a large amount of lactic acid, which is a degradation product, is generated. In particular, in the case of dairy cows, it frequently occurs within one month after calving when milk yield increases, and it is such a common disease that about 20 out of 100 farms experience it.

Conventionally, in order to effectively prevent lactic acid generated in the rumen, a period of at least 3 to 4 weeks was required, and the contents of the rumen had to be removed or neutralized. To neutralize the rumen, 500 g (based on an adult cow of 450 kg) of magnesium hydroxide or magnesium oxide was mixed in 10 liters of warm water and administered orally, and then the abdomen was well massaged to manage the cow with acidosis.

In addition, in order to prevent rumen acidosis, feeding a large amount of concentrated feed was not recommended, and feeding of concentrated feed was restricted, and roughage was provided. When changing feed, an adaptation period of about 7 days was given for the feed transition, and in order to maintain the rumen pH, an appropriate amount of roughage was first fed, and the level of crude fiber was adjusted to about ADF 18-19% and NDF 25-28%.

In the case of concentrated feed, unlike roughage, the content of protein and fat is high, and although a large amount of administration is necessary to improve the quality of the raised individual, the large administration of concentrated feed was limited due to the risk of rumen acidosis caused by large administration of concentrated feed. In addition, the conventional treatment method for rumen acidosis had the limitation that the process was cumbersome and the treatment period required more than 3 weeks, consuming a lot of time, and it was difficult to maintain the commercial value of the individual.

Accordingly, there is a need for a bacteriophage having lytic activity against Streptococcus bovis/Streptococcus equinus complex (SBSEC) and lactic acid-producing bacteria that cause rumen acidosis, and capable of controlling the biofilm generated by said strains, or a feed additive using the same.

### DETAILED DESCRIPTION OF THE INVENTION

### Technical Problem

The inventors of the present invention obtained a bacteriophage vB_SbRt-pBovineB21 and confirmed that it has lytic activity and biofilm control activity against Streptococcus bovis/Streptococcus equinus complex (SBSEC) and lactic acid-producing bacteria.

Accordingly, it is an object of the present invention to provide the bacteriophage vB_SbRt-pBovineB21.

Another object of the present invention is to provide a composition for inhibiting proliferation or killing of SBSEC and/or lactic acid-producing bacteria.

Another object of the present invention is to provide a composition for treatment, prevention, suppression, or improvement of rumen acidosis.

Another object of the present invention is to provide an antibacterial agent comprising the bacteriophage vB_SbRt-pBovineB21 as an active ingredient.

Another object of the present invention is to provide a disinfectant comprising the bacteriophage vB_SbRt-pBovineB21 as an active ingredient.

Another object of the present invention is to provide a feed additive for ruminants. Another object of the present invention is to provide a feed composition for ruminants. Another object of the present invention is to provide a method for treatment or prevention of rumen acidosis in ruminants.

### Technical Solution

The present invention relates to a bacteriophage vB_SbRt-pBovineB21, and the bacteriophage according to the present invention exhibits excellent lytic activity and biofilm control activity against Streptococcus bovis/Streptococcus equinus complex (SBSEC) and lactic acid-producing bacteria.

The present invention will be described in more detail hereinafter.

One embodiment of the present invention is a bacteriophage vB_SbRt-pBovineB21 having lytic activity and biofilm control activity against Streptococcus bovis/Streptococcus equinus complex and lactic acid-producing bacteria.

As used herein, the term “Streptococcus bovis/Streptococcus equinus complex (SBSEC)” refers to a group of Gram-positive streptococci derived from humans or animals, which is regarded as a causative factor related to acute ruminal lactic acidosis in ruminants, and the said group includes lactic acid-producing bacteria. SBSEC was conventionally used to refer to Streptococcus bovis and Streptococcus equinus, but its meaning has now changed, and depending on the identity of the strains, it is used to refer to a group of strains classified into four clades, namely Streptococcus ruminicola, Streptococcus equinus, Streptococcus lutetiensis, Streptococcus bovis, Streptococcus infantarius subsp. infantarius, Streptococcus gallolyticus subsp. gallolyticus, Streptococcus gallolyticus subsp. macedonicus, Streptococcus gallolyticus subsp. pasteurianus, and Streptococcus alactolyticus.

As used herein, the term “bacteriophage” refers to a virus that infects a specific target bacterium. The head of the bacteriophage, which is surrounded by a protein capsid, consists of a nucleic acid genome, and some bacteriophages have tails composed of lipids or proteins. Most bacteriophages contain double-stranded DNA as genetic material. The bacteriophage vB_SbRt-pBovineB21 of the present invention has been confirmed to possess killing ability that targets and kills Streptococcus bovis/Streptococcus equinus complex and lactic acid-producing bacteria.

Bacteriophages can be found in all environments inside or outside water, soil, and plants. Bacteriophages attach to target bacteria and inject their DNA into the host, replicate themselves inside the host, and cause the death of the bacteria. Bacteriophages are relatively safe for animal or plant cells, and do not affect other beneficial microorganisms. In addition, bacteriophages are relatively easily isolated from host bacterium-containing environments.

As used herein, the term “lactic acid-producing bacteria” is a generic term referring to bacteria that produce lactic acid in their metabolic process, and in the present invention, the lactic acid-producing bacteria may include Lactobacillus and Lactococcus strains.

As used herein, the term “lysis” refers to a phenomenon in which the cell wall or cytoplasmic membrane of bacteria is destroyed and the bacteria are lysed, meaning that the bacteria are killed.

As used herein, the term “biofilm” refers to an aggregate of microorganisms in which microbial cells adhere to each other on an attached surface. Microorganisms form microbial films in response to various factors, examples of which include recognition of specific or nonspecific attachment sites on a surface, nutritional signals, or, in some cases, when sessile protozoa including planktonic protozoa are exposed to the environment.

As used herein, the term “killing ability” refers to the ability to kill or eliminate living organisms. In the present invention, it may mean the ability of a bacteriophage to kill strains.

In the present invention, the bacteriophage vB_SbRt-pBovineB21 may consist of, or comprise, the nucleotide sequence of SEQ ID NO: 1.

The sequence used in the present invention is to be interpreted as including sequences

showing substantial identity with the sequence disclosed in the sequence listing, considering biologically equivalent variants.

As used herein, the term “substantial identity” means a sequence showing at least 60% homology, more specifically 70% homology, even more specifically 80% homology, and most specifically 90% homology, when aligned to the sequence of the present invention and any other sequence so as to correspond as much as possible, and analyzed using algorithms commonly used in the art.

Therefore, in the present invention, a bacteriophage consisting of, or comprising, a nucleotide sequence having high homology of at least 60%, at least 70%, at least 80%, or at least 90% with the sequence represented by SEQ ID NO: 1 is also included within the scope of the present invention.

The bacteriophage vB_SbRt-pBovineB21 according to the present invention is a phage having genetic difference from conventionally known bacteriophages, was isolated from the rumen of ruminants in Korea, and was confirmed morphologically to have a morphology similar to that of Podoviridae through transmission electron microscopy (TEM). In addition, as a result of genome analysis of the bacteriophage according to the present invention, it was confirmed to have low homology with existing bacteriophages, and it was confirmed that the genome is distinguishable from other phages (see Example 4).

Specifically, the bacteriophage according to the present invention infects host bacteria selectively, and therefore has the ability to lyse only the host bacteria without causing other damage in the body of ruminants or humans. Specifically, the bacteriophage according to the present invention exhibits lytic activity also against lactic acid-producing bacteria in addition to SBSEC isolates, and it has been confirmed to have excellent biofilm control ability against SBSEC and lactic acid-producing bacteria (see Examples 2 and 5).

In addition, the bacteriophage according to the present invention exhibits excellent environmental stability, and it is able to survive even in an acidic environment of pH 5 or less. Accordingly, it has been confirmed that it can grow stably in the rumen of ruminants (see Example 3-1). At the same time, it exhibits excellent lytic ability against host strains, and excellent host adsorption ability and growth ability, so that it has been confirmed that a large number of host strains can be infected within a short time (see Examples 3-2 to 3-3).

The inventors of the present invention named the bacteriophage obtained as vB_SbRt-pBovineB21, and deposited it at the Korean Culture Center of Microorganisms (KCCM) on Aug. 13, 2021, with the deposit number KCCM 13031P.

In one embodiment of the present invention, SBSEC may include one or more selected from the group consisting of Streptococcus ruminicola, Streptococcus equinus, Streptococcus lutetiensis, Streptococcus bovis, Streptococcus infantarius subsp. infantarius, Streptococcus gallolyticus subsp. gallolyticus, Streptococcus gallolyticus subsp. macedonicus, Streptococcus gallolyticus subsp. pasteurianus, and Streptococcus alactolyticus.

In one embodiment of the present invention, the lactic acid-producing bacteria may include Lactobacillus strains and Lactococcus strains. Specifically, the lactic acid-producing bacteria may include one or more strains selected from the group consisting of Lactobacillus plantarum, Lactococcus lactis, Lactobacillus casei, and Lactobacillus sakei.

Another embodiment of the present invention is a composition for inhibiting proliferation or killing of SBSEC and lactic acid-producing bacteria, comprising the bacteriophage vB_SbRt-pBovineB21 as an active ingredient.

In the present invention, the composition may further comprise an additional antibacterial agent.

Another embodiment of the present invention is a composition for treatment, prevention, suppression, or improvement of rumen acidosis, comprising the bacteriophage vB_SbRt-pBovineB21 as an active ingredient.

As used herein, the term “rumen acidosis” refers to a dyspeptic disease occurring due to excessive production of lactic acid in the rumen caused by a sudden change of feed or excessive feeding of concentrated feed during management of ruminants, and means a disease in which the number of starch-degrading bacteria increases rapidly and a large amount of lactic acid, which is a degradation product, is produced when a large amount of feed containing starch is ingested.

As used herein, the term “active ingredient” means that the bacteriophage according to the present invention is included in an effective amount to achieve the intended effect, for example, a therapeutic effect or antibacterial effect for rumen acidosis.

As used herein, the term “treatment” means any act in which the symptoms of rumen acidosis are improved or favorably altered by administration of a composition comprising the bacteriophage according to the present invention.

As used herein, the term “prevention” means any act in which the symptoms of rumen acidosis are suppressed or delayed by administration of a composition comprising the bacteriophage according to the present invention.

The composition according to the present invention may further comprise known carriers or additives pharmaceutically, food, or feed acceptable, in addition to the strains included as active ingredients. The composition comprising the bacteriophage vB_SbRt-pBovineB21 of the present invention may further comprise, in order to prevent quality deterioration, binders, emulsifiers, preservatives, etc. to be added, and in order to increase efficacy, amino acid preparations, vitamin preparations, enzyme preparations, flavoring agents, non-protein nitrogenous compounds, silicate preparations, buffering agents, extractants, oligosaccharides, etc. to be added to feed. In addition, it may further comprise feed mixing agents and the like, but is not limited thereto.

Another embodiment of the present invention is an antibacterial agent comprising the bacteriophage vB_SbRt-pBovineB21 as an active ingredient.

Another embodiment of the present invention is a disinfectant comprising the bacteriophage vB_SbRt-pBovineB21 as an active ingredient.

As used herein, the term “antibacterial agent” means a substance that kills or inhibits the growth of microorganisms, and is a generic term for preservatives, bactericides, and antibiotics.

As used herein, the term “disinfectant” means an agent intended for sterilization of pathogenic bacteria.

Another embodiment of the present invention is a feed additive for ruminants comprising the bacteriophage vB_SbRt-pBovineB21 as an active ingredient.

Another embodiment of the present invention is a feed composition for ruminants comprising the bacteriophage vB_SbRt-pBovineB21 as an active ingredient.

The composition according to the present invention may be manufactured in a feed form, and at this time, the feed is not particularly limited, and may be any feed such as powdered feed, solid feed, moist pellet feed, dry pellet feed, extruder pellet feed, or raw feed. The bacteriophage vB_SbRt-pBovineB21 of the present invention may be manufactured in the form of a feed additive and mixed into feed, or may be manufactured by directly adding it into feed.

The feed additive of the present invention may further comprise known carriers or stabilizers pharmaceutically, food, or feed acceptable, in addition to the active ingredient, and may further comprise, as necessary, various nutrients such as vitamins, amino acids, minerals, antioxidants, and other additives. Its form may be a suitable state such as powder, granule, pellet, suspension, etc. When supplying the feed additive of the present invention, it may be supplied alone to a unit animal or mixed into feed.

Another embodiment of the present invention is a method for treatment or prevention of rumen acidosis in ruminants, comprising the following step:

Administering the bacteriophage vB_SbRt-pBovineB21 to a ruminant.

As used herein, the term “ruminant” refers to a herbivorous animal having a stomach divided into 4 or 5 chambers, found in Camelidae, Tragulidae, Cervidae, Giraffidae, and Bovidae, and also called a ruminant animal. In the present invention, the ruminant may be a herbivorous mammal such as cattle (bovine), sheep, goat, camel, or bovid, and the composition according to the present invention may be administered to ruminant mammals producing milk (or dairy), for example, cattle, dairy cows, sheep, or goats.

Another embodiment of the present invention is a method of lysis of SBSEC and/or lactic acid-producing bacteria comprising a step of treating with the bacteriophage vB_SbRt-pBovineB21.

### Advantageous Effects

The present invention relates to a bacteriophage vB_SbRt-pBovineB21 having excellent lytic activity and biofilm control activity against Streptococcus bovis/Streptococcus equinus complex (SBSEC) and lactic acid-producing bacteria, and the phage and/or composition according to the present invention can be used as an antibacterial agent, a disinfectant, and a feed additive for ruminants.

### DESCRIPTION OF THE DRAWINGS

FIG. 1 is a transmission electron microscopy (TEM) photograph confirming the morphological characteristics of the bacteriophage vB_SbRt-pBovineB21.

FIG. 2 is a graph showing the results of testing stability of the bacteriophage vB_SbRt-pBovineB21 at various temperatures (left) and pH values (right).

FIG. 3 is a graph showing the results of testing adsorption ability (left) and one-step growth curve (right) of the bacteriophage vB_SbRt-pBovineB21 against host bacterium S. ruminicola.

FIG. 4 is a drawing showing the results of testing host control ability of the bacteriophage vB_SbRt-pBovineB21 at different multiplicities of infection (MOI) against host bacterium S. ruminicola.

FIG. 5 is a drawing showing a whole-genome map of the bacteriophage vB_SbRt-pBovineB21.

FIG. 6 is a drawing showing the results of phylogenetic tree analysis based on the whole-genome sequence of the bacteriophage vB_SbRt-pBovineB21.

FIG. 7 is a drawing showing a heat map based on the whole-genome sequence of the bacteriophage vB_SbRt-pBovineB21.

FIG. 8 is a drawing showing a phylogenetic tree based on the DNA polymerase sequence of the bacteriophage vB_SbRt-pBovineB21.

FIG. 9 is a drawing showing a phylogenetic tree based on the major capsid protein sequence of the bacteriophage vB_SbRt-pBovineB21.

FIG. 10 is a drawing showing the results of comparison of major proteins of bacteriophage C1 belonging to the genus Fischettivirus, which shows high homology with the bacteriophage vB_SbRt-pBovineB21.

FIG. 11 is a graph showing the results of analysis using crystal violet of the ability of the bacteriophage vB_SbRt-pBovineB21 to inhibit biofilm formation of S. ruminicola depending on concentration, over time.

FIG. 12 is a photograph showing the results confirmed with confocal laser scanning microscopy (CLSM) of the ability of the bacteriophage vB_SbRt-pBovineB21 to inhibit biofilm formation of S. ruminicola over time.

FIG. 13 is a drawing showing the results confirmed with crystal violet (left) and confocal laser scanning microscopy (right) of the ability of the bacteriophage vB_SbRt-pBovineB21 to inhibit biofilm formation against various host bacteria.

### BEST MODE

Bacteriophage vB_SbRt-pBovineB21 having lytic activity and biofilm control activity against Streptococcus bovis/Streptococcus equinus complex (SBSEC) and lactic acid-producing bacteria.

### MODE FOR CARRYING OUT THE INVENTION

Hereinafter, the present invention will be described in more detail with reference to the following Examples. However, these Examples are only for illustrating the present invention, and the scope of the present invention is not limited by these Examples.

### Example 1: Isolation and Morphological Analysis of Bacteriophage

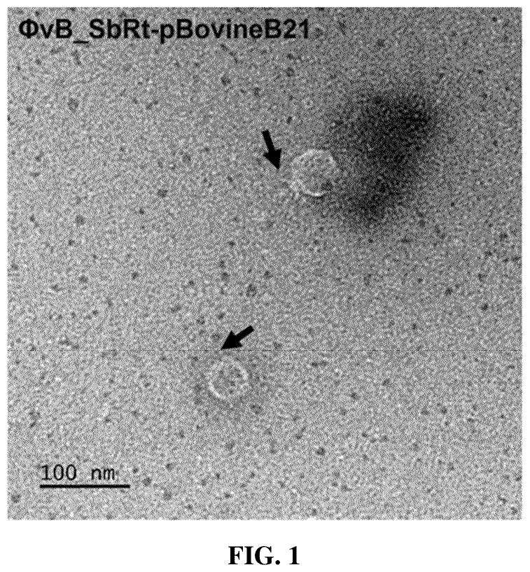

Rumen fluid of domestic ruminants was collected, and after shaking culture of S. ruminicola KCTC 43306, as shown in Table 1, as a host bacterium at 37° C. for 24 hours, the culture was centrifuged at 10,000×g for 20 minutes, and the obtained supernatant was filtered through a 0.45 μm filter. The obtained filtrate was used to isolate a lytic phage by the double-layered agar (DLA) method. The obtained phage was named vB_SbRt-pBovineB21 according to the nomenclature of bacteriophages. For morphological analysis of the isolated phage, observation was performed with transmission electron microscopy (TEM), and as can be confirmed in FIG. 1, it was confirmed to have a morphology similar to that of Podoviridae in morphological classification of bacteriophages.

### Example 2: Host Range Analysis of Bacteriophage vB_SbRt-pBovineB21

The host range of the phage vB_SbRt-pBovineB21 was analyzed using a total of 64 strains, including 51 SBSEC isolates previously isolated by the inventors of the present invention, 8 standard strains of Streptococcus bovis/Streptococcus equinus complex (SBSEC), S. agalactiae, 3 species of Lactobacillus, and 1 species of Lactococcus. The presence or absence of plaque formation against each strain was confirmed by the above-described double-layered agar method, and the results are shown in Table 1. As a result, as can be confirmed in Table 1 below, the bacteriophage vB_SbRt-pBovineB21 was confirmed to be a phage having lytic activity also against lactic acid-producing bacteria in addition to SBSEC isolates.

TABLE 1

| | | Phage sensitive bacteria | |
|---|---|---|---|
| Strain | Source | vB_SbRt-pBovineB21 | |
| S. ruminicola isolates | Capra aegagrus hircus | 1/1 | (100%) |
| | Bos taurus | 0/2 | (0%) |
| S. equinus isolates | Bos taurus | 2/13 | (15.4%) |
| | Bos taurus coreanae | 6/17 | (35.3%) |
| | Capra aegagrus hircus | 9/14 | (64.3%) |
| S. lutetiensis isolates | Capra aegagrus hircus | 4/4 | (100%) |
| S. equinus ATCC 9812^{T} | Equus ferus caballus | 0/1 | (0%) |
| S. bovis ATCC 33317^{T} | Bos taurus | 0/1 | (0%) |
| S. infantarius subsp. infantarius | Homo sapiens | 0/1 | (0%) |
| ATCC BAA-102^{T} | | | |
| S. gallolyticus subsp. gallolyticus | Phascolarctos cinereus | 0/1 | (0%) |
| DSM 16831^{T} | | | |
| S. gallolyticus subsp. pastuerianus | Homo sapiens | 0/1 | (0%) |
| NEM 1202^{T} | | | |
| S. gallolyticus subsp. macedonicus | Cheese | 0/1 | (0%) |
| DSM 15879^{T} | | | |
| S. lutetiensis NCTC 13774^{T} | Homo sapiens | 0/1 | (0%) |
| S. alactolyticus ATCC 43077^{T} | Sus domesticus | 0/1 | (0%) |
| S. agalactiae KCCM 11957^{T} | Homo sapiens | 0/1 | (0%) |
| Lactobacillus plantarum subsp. | Cabbage | 1/1 | (100%) |
| plantarum KCTC 3108^{T} | | | |
| Lactococcus lactis subsp. lactis | Unknown | 1/1 | (100%) |
| KCCM 41572^{T} | | | |
| Lactobacillus casei KCCM12452^{T} | Cheese | 1/1 | (100%) |
| Lactobacillus sakei KCCM 40264^{T} | Yeast starter | 1/1 | (100%) |

This shows that the phage vB_SbRt-pBovineB21 has the potential to control lactic acid-producing bacteria in addition to SBSEC, which are causative agents of acidosis.

### Example 3: Biological Characteristics Analysis of Bacteriophage vB_SbRt-pBovineB21

### 3-1. Temperature and pH Stability

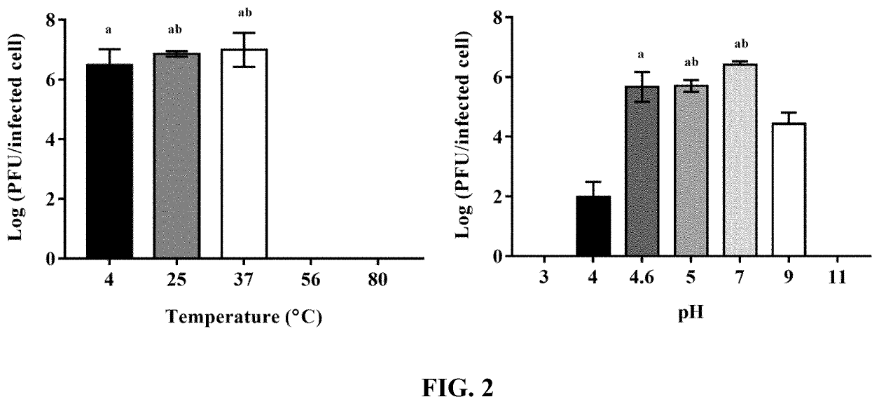

In order to verify the environmental stability of the bacteriophage vB_SbRt-pBovineB21, analysis was conducted under various ranges of temperature and pH conditions. The phage filtrate was exposed for 3 hours under each condition, and plaque-forming units (PFU) were investigated. As can be confirmed in FIG. 2, the phage was stable at temperatures of 4 to 37° C. and pH values of pH 4 to 9. This allows determination that the phage vB_SbRt-pBovineB21 is not affected by a wide range of temperature and pH changes, and is available for long-term storage and utilization. In addition, survival ability at a pH condition of pH 5 or less, which is the condition of rumen acidosis, was confirmed.

### 3-2. Analysis of Host Adsorption Ability and Growth Curve

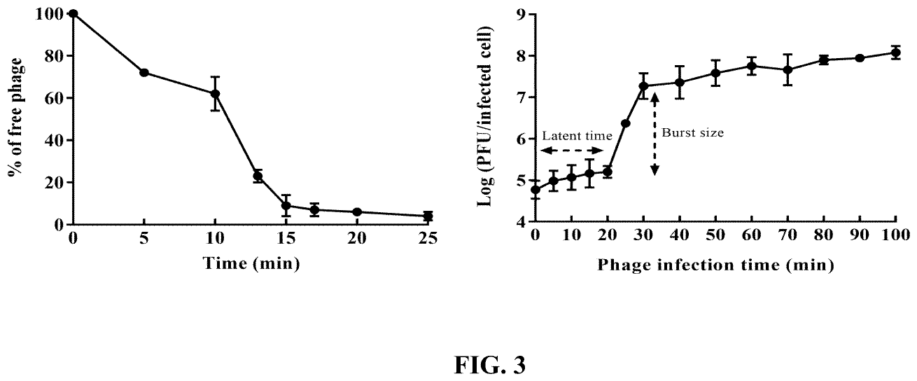

The adsorption ability (adsorption) and one-step growth curve analysis of the phage vB_SbRt-pBovineB21 against host bacterium S. ruminicola were conducted. As can be confirmed in FIG. 3, when PFU/mL was measured over time, it showed a maximum adsorption rate of 90% or more within 15 minutes, and after undergoing about 20 minutes of a latent period, it was observed that 367 phage virions were produced per infected bacterial cell. This shows that the phage vB_SbRt-pBovineB21 can infect a large number of bacteria within a short period of time.

### 3-3. Analysis of Lytic Ability

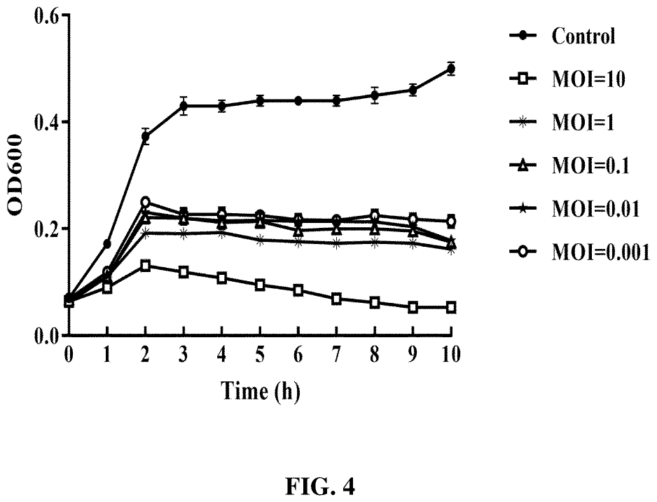

The host control ability of the phage vB_SbRt-pBovineB21 against S. ruminicola was confirmed according to various multiplicities of infection (MOI). As a control group, a host bacterium culture without addition of phage was used, and absorbance at 600 nm was measured using a spectrophotometer every 2 hours up to 10 hours. As can be confirmed in FIG. 4, a distinct difference compared to the control group was observed from low MOI values to high MOI values. This shows that the phage vB_SbRt-pBovineB21 effectively inhibits the proliferation of host bacteria for a long time and also has high lytic activity even at low concentrations.

### Example 4: Genetic Characteristics Analysis of Bacteriophage vB_SbRt-pBovineB21

The genome of the bacteriophage vB_SbRt-pBovineB21 was extracted, and analysis of gene sequence and genetic characteristics was performed. The whole-genome sequence was decoded using the Illumina HiSeq platform, and annotation of each open reading frame (ORF) was analyzed using the RAST (Rapid Annotations using Subsystems Technology) server. The sequence of the bacteriophage vB_SbRt-pBovineB21 is shown in Table 2 below.

TABLE 2

| Sequence | | | |
|---|---|---|---|
| No. | Name | Sequence listing (5′->3′) | Remark |
| 1 | VB_SbRt- | ATTAATGTATACTCTGTAGAACTTCTAGACAC | |
| | pBovineB21 | TAAAGAGCATGTATATATGGAGTACGCTTTAT | |
| | | TAGCTGACAACATGAAATCAGCTATTGAATGT | |
| | | GCTAAAGAAAAAGCTTACAACATGATTAGTGA | |
| | | AAACAAAATTGTTATTCGTTATGTTTCAGTAC | |
| | | AAGACGGCAACGAACAATATGTATACAATGTA | |
| | | TTTACTAAAAAACTTGAAAAATGGTATTAAGG | |
| | | AGCAACAACATGAACAAACAAGAATTAATTAA | |
| | | AGATATTGAATTTAAAAAAATAGACGATGTGT | |
| | | TTACTGATTTATCAAATGGATATAATTTAGGA | |
| | | TTATCCACAGCAATTGAAGTTATAAAAGAATT | |
| | | TAACGACACTGAACAAGAGTTAACCGTTGATT | |
| | | TTAAACAACAACAGCCTTTTGTGTCTTCCACT | |
| | | CCACCTCACTACCAGGGCACAATCCAACCTAT | |
| | | TGACCTCATCAACGCTCAAGACCTTAACTTTA | |
| | | ACCTTGGAAACGTTGTTAAATATGTCTGTCGT | |
| | | GCTGGTAAAAAACAAGGCGAGAACATCTTAAC | |
| | | TGACCTAGAAAAAGCAAAAGATTACATCAACT | |
| | | TTGAAATTGAAAGAGTAAAACAATGAATGAAA | |
| | | TAAAACTAAAGTTATTAGTTAACTTATTACTA | |
| | | TTATTTTTATGGTCAATATATTTTATATTTAA | |
| | | AGGTAAAACCAAATGAACAAACGTCAACGCAA | |
| | | AAAACTAGCAAAAAAACGAGCTATGAAAACAG | |
| | | GTAGTTATACTCCCAGAAATACTTTTCGCTCT | |
| | | ACTATGTCTATCAAATCAACATTATTCGACTC | |
| | | TAATCAACCAAAAAATGAAAAAATGATATATC | |
| | | TAGCTGAAAGAAACTTAAACAGACTAGCATTT | |
| | | GTTATCACAAAACCAAATATCGTTAATCTAAC | |
| | | AAACCTAACAGAAGATGACGTTAAATATATTC | |
| | | AATCACCAAAATTTACTCAAACAGTATCTAAC | |
| | | ATACAAGAATACTTTAACTATGACCGTGCTAG | |
| | | ATACCGAGACCCAGAACGAGTTGATAACATTA | |
| | | CACGTCACGTCATGACCAAATACTTCTATCAA | |
| | | CGTGATATTGAAAGCACACGTGCACTAGATAT | |
| | | TGCTATCAAACGACTAACAAAACAACTAGAAA | |
| | | AAATAACTAAAAAATACTCAAAAGGAAAAGAC | |
| | | AGACGTGCAACAAATAATCCGTTTGTTATCGT | |
| | | CTATTACCAAGACTTGGAGCTAAGATAATGAA | |
| | | ATATTTAATTACATATCACACAAAACTAGACA | |
| | | ACGGCTATATTGATGAACGTCAAATCATTTGT | |
| | | AAGAACAAATACAAAGCTAAAAGAATTTATGA | |
| | | CAAGTTAGCGGGAAAAAAGCGAACTATCAATA | |
| | | TTGAATTAGTCGAGGTATAAACATGACCACTA | |
| | | CTACTTCCACTTACCGTGTGTCAATCAACTAT | |
| | | AAAAGCAAAGTTATTTTACTTTCAAAACCTTT | |
| | | GAAAGAATGCAAATACTTTCACAATTATGCCA | |
| | | ATTATATTGACACTACTTATAAATTAAATAAT | |
| | | CAAGTTGTAAATGTACACTTTGATACTAAAAA | |
| | | TCACCGTGTGAACCATTACACACCGAATTATA | |
| | | AAACAGCTAAACTTTTATTTGAAAAACTTAAA | |
| | | AACACGAAAAAAGATTACTTTTTTTTCAATTC | |
| | | TTTAGATAATTAGCTTGACAAACAAAAAACAA | |
| | | CTATTATATAATTAACTTACAAACATGAAAGG | |
| | | AGCCAAACACATGGCATTACAATTTAACACAA | |
| | | TCGGATTTATCAACTTCAACAAAGATTACAAC | |
| | | AAAGTTTTGAAGTCAGGAGCTATCACAGTTTC | |
| | | ATTCACTTCTACTATTAAAAATAAAGACGGTG | |
| | | AATATGAAAAACAATATTTCAATGGTCTTATC | |
| | | CCTGCTAAACTAGCACAACACGTTAAACCATA | |
| | | CCTAAATAAAGAACTTGTAAAAATTCATGGTA | |
| | | TTGCTTCTCCTGGTCAAAAAGGATATATCAAC | |
| | | TTTACAATTCTAGAAGTTGAACAATATAAAAA | |
| | | AGAAACAAAATCAGACGTGAATGACAATGACT | |
| | | TACCATTCTAAAGGAGGTGACGGGAGGTGAAT | |
| | | AGCCTCCCTTTTATTTTATGGCTAAAAAGAAA | |
| | | ACATTACCAAAAACACTAAAACAAAAAGCTGT | |
| | | ATCACTTTTTGCAACCGAAACAGAAGAATATA | |
| | | CTTATTATTTAAACGAATACCGCTCAAAATAC | |
| | | CTACCACCTGAATTTAACCAATTAGAACTGTT | |
| | | AGATGAACTTTGTAACGAAGAACTCGACCATT | |
| | | ATATGAGCGTCACCACACGTGGTGATGGTAAA | |
| | | TCATTTAACTATATTTCAGCGGTTGGCTACCT | |
| | | CTGCTATCACCTTAACATGGGCTGTACTTTAC | |
| | | TTGTCCGCCACTTTACTTTACAAGACAAAATG | |
| | | AGAGAACTTGTTGAAGACATCTTACAAACTAT | |
| | | CGGCTGGTGTAATTTCTCTACAGATTACCACT | |
| | | ACCGCTCTACTTCAGATTATTTAATCATTTCT | |
| | | ATTGGTGACAAAGACGTTTTTCTTATCACTGA | |
| | | CATAAACAACGCTAGCGACTTGAAACAGTCTT | |
| | | CTGCTGTTTTGAAAAAATTTCCTATCATGCTA | |
| | | TACGATGAATTTCTAACATTACCAGATGATTA | |
| | | TTGTAAAAACGAGTACGAAAAAATTCGCACCA | |
| | | TTTACAAATCTATTGACCGTGTTAAAAACCGT | |
| | | CCGTATATCAAAACACCTAAAATGATTTATCT | |
| | | AGCTAACCCAGTTAACTTTGATAGTCCACTAC | |
| | | TACCATCACTAAAAATCTATAACAAATTACAA | |
| | | ACACAAGAAATTAACACAATTAAACAATATAA | |
| | | CAACGTCTTGTTAGAACTACGCCGAAACGATT | |
| | | CACGCAATGACGGAAAGAACCTACGTGCCTTT | |
| | | CCTGATGGTGATGACGCTGACGTTACTGGTGA | |
| | | ATTTCAATTCTCAAATCACAAGCTGGTTTCCG | |
| | | AAGAAACCTATTTTAATATCTATAACCACGCA | |
| | | AAATCAGTAAAAGTTAAATTAGACGATAAATT | |
| | | AATGTTGCATATCATTGCTAAAAATGATAATA | |
| | | TAGTATTATCAATAGAAAGAACAGATAACACT | |
| | | GAAGAATATTGTATAAATTTAGTTGATGAAAC | |
| | | AGAAAAAAGCAAGTATTTAAACGATAAATACT | |
| | | ACAAACAAAGCTTCATCAAGAAACATGAAAAA | |
| | | GGTATGTTTCTTTACAAGGACAGCTTTTCAAA | |
| | | ATCTTATATGGAACGTGACACAATGCTTATGC | |
| | | AACTAAACTTGTTTAAGCTTATCCCGACAGCT | |
| | | AAAGAAATTACAACTGAAGAAACATATATCAA | |
| | | AATCAAGGAAAACAATTTCCTTAAATCTCTAG | |
| | | CTCAAAAATATGAATAATAAAGGAGTCTTAAT | |
| | | ATCATGCACAATCAAAATGAACTACTTGACTT | |
| | | TTTAAAAAAGTTTAAAGGAAAGAAAGTCTCAT | |
| | | TATATGCTGATATTGAGACTTTTACTTGTAAC | |
| | | AAAATTGAAGGCAGTGAACACCCAACAAAATA | |
| | | CCATTCTTTTACATACTCACTAGCCATTGCCT | |
| | | ATTTTAATGATAGCGACTTTCCAAAAGTAGCA | |
| | | GTATTTAATAATTTTTATGATTTTTTTGAAAA | |
| | | AGTAAAAGAAAGAAAAATCAGAAAATCGCTTT | |
| | | CTTTCGATTTTGTCTTTCACAATGGCGAAAAA | |
| | | TTCGATAACCACTTTTTTATCGAAGAAATGCA | |
| | | AGCTTACTATGGTTTACCAGTTTACACTGAAT | |
| | | ACAACAAAAACGCAAATAACCTAGCTAACGAA | |
| | | CACGCTAGAAAAATGTCTACCATTGATACTGA | |
| | | AGAAAAAAAACACGGTCTTGTTTTGGAGAGTC | |
| | | GTGTAAAATCTTCTAACAACGTATCTGTTAAA | |
| | | GCGTTCGTTCATGGCAGACGTGTTGAATTTAT | |
| | | TGACTCCTTTAAAAAAATGAACACATCTATCG | |
| | | CTGTACTCGGTAAAATGTTACTTAACAATAAT | |
| | | TTAATCACTGAAGAATATTTGAAAACCGATTT | |
| | | TGACTATCAATGTTTTGATAAAGATGAAGACA | |
| | | TTGAAAGAGAGCTAGTTAAAACATATGTTAAG | |
| | | CGTTGTTTTGAAAGTCTTAATGAAAAACAAAT | |
| | | GATTTATATCAGAAATGACGTTATTATTTTAG | |
| | | CATTAGGAGTTAAACATTATAAAACCTTATTC | |
| | | TATGGCTTTGACTTTTCTAAAATAACCTTTAC | |
| | | TCAAAATATCAAAGAGGAATACTCAAAATATA | |
| | | ACAAACTAGCTGAATTTCAACTATTAAAAACA | |
| | | GACGGTCGCTTCTCTCATTTAAAATTAAACGA | |
| | | TTATCAAATATGTGGCATGTCTGGTTTTGACT | |
| | | ATTTCCGTTCATATTACAAAGGCGGTTTAAAC | |
| | | CTATACAATGATAAATACATAGGAAAAATATT | |
| | | AAATAGAGACGGTTTTTCTATTGATTTAAACA | |
| | | GTTCTTATCCTACAGTCATGTATAAGGAGAAG | |
| | | TTACCAACATATTTAATAGCTTTAAACGAAAA | |
| | | AAAATCAATTGCCAATTTTGACTATAACAACA | |
| | | ATAATATTATGTCGTTCTTTACAATGACAATT | |
| | | GAAAACGCTAATAAGTATATACTTTCTAAAAT | |
| | | AGAAAGTAAAGTCTTAAGAAATGCAATCGTTA | |
| | | AATACTACAATTCAAAAAATGGTCTTGTTTAC | |
| | | TATAACACCGTTCTATTAAGACTACTATCTAA | |
| | | AATTACAAAGAAAGACTTTAAGTCACTTCCAG | |
| | | TTGAAAGCACTGCTACATTTCAATGTGAATAC | |
| | | TTTGGAGCACGTGACGTGATTGCCCGTAACTA | |
| | | TTTTATTAAATCACAAGGGAAAATGAAAAACA | |
| | | AACTGGCTTGTGAGATTGATACAATCGACCCC | |
| | | CTAAACATAGAAATGACTAATGAACCAAAACC | |
| | | AGCTAAATATAATTTTAGTGATGAAATGGTAC | |
| | | AAGGTTCAAAAGTCCTTTTAAATGGGATTTAC | |
| | | GGTGTTCCAGCTTTACGTATTCATTTTGATAT | |
| | | TTTTAAACGTGTCGGTAACGACTTTGAAAACG | |
| | | TTAAAAACGGCTTTACCAATAAAGAAAGAAAT | |
| | | ATTGTTTTTTCTGCTGGTGTTACTGCTTTCGC | |
| | | TTTTCATAATCTACTATCACCACTTCAGTATT | |
| | | TAACACCCAAAGAAATTGATGAATATTTCTGG | |
| | | TATGCTGATACTGATAGTCTTTACATGGATAA | |
| | | ACGAGCACTTGACAAGTTTCCTAAACCAATGT | |
| | | TTCATAAAATGAATTTGGGGGGTTGGGATATT | |
| | | GAACACGAAAACATCACTAAATTTTATGCATT | |
| | | CAATCATAAAAAATATTGCTTGTATGATAACG | |
| | | GTATTGTTGTCCGTTGCGGTGGGGTATCAAAA | |
| | | TCACTTATTAAAGAATGGATAAAACATTCACA | |
| | | TGATGACTTTGAATTTTTTGTGAAGTGTTATT | |
| | | TTTCAGATGGAACTATTGTTTCATCAACACGT | |
| | | TCTATCAGGAACGAATATAACACTATCTCTAT | |
| | | TTATAATTCATCAGCAAAACTTGAAAAAGGTT | |
| | | TTCCTTACTTTGATAGCTACATTTTACAAAAT | |
| | | GAAAAAGAGCTAGAAAAAATAAAATCACAAAT | |
| | | ACGTGATGAAATAGCTAACCAAAACTCAAACG | |
| | | AGCTTTTATATGTTGAAACACCTTATGGTTCA | |
| | | ATTGGTTCTAATGAAATAATTCCAAATGAAGA | |
| | | CGTGCCAGAAAATAATAACATTCGTGAACTAA | |
| | | TCGAAGAATATAACAATTTTAAAAGACACAAT | |
| | | CAAAAGGACTTATTCTAAAATAAGCCCTTTTT | |
| | | CTCATATAATAAATTGGTTGAAAAGTCACACC | |
| | | ATTCAAGGTTATACCAAGCGTTATTAGTCTTG | |
| | | TCATGGTCTATGTGATGAATGGTATTATAATT | |
| | | GTTTGGATTCGGTATGAACGTTTCAGCAACTA | |
| | | GCCTATACACGTACACTGTTTTATATAAACCG | |
| | | TTTTGTGATAAGTTAACCCTTTCACGTCCACC | |
| | | CTTATCGTATCTAGTTTTTAAGATTTTACCAG | |
| | | TTTTCTTACTTTTAACAAAGCCACCATTTTGA | |
| | | ATTAAATAGTTGGTAAAAATCGGGTGTTCTTT | |
| | | CCATTCAATAGATACACTTTTCAAATCGTTTA | |
| | | ACATCTTTTTCACTAATGTTTAGTTCAGGTAA | |
| | | ACCAAATTTTTCTTTCAAAGCAATAACAGTCT | |
| | | TAATGTCAGCAATATCTAATCGTTTGCCATTC | |
| | | AAACACGCACGATATCCATAATTAGTGTCAAA | |
| | | ATATAAACAGTCGTATGGATAGCGCTTGTCTT | |
| | | CAATATTGAAATTAATCTCACTCATGTTCTTT | |
| | | CTCCTTAATCATTTTTAAAATTGCAGGGTCTA | |
| | | CAGTTTTAACTGTTTCCTCTTTAACAACTTCA | |
| | | GTTTTTCCTAATTGGTCATTATCAAGCATGTT | |
| | | TTTAAGTTTCTCAGGCACAGGAATGAACACGC | |
| | | TAGCATTTTCAAGAATAGAAATAATTTCCATA | |
| | | ACAAGATAGTATCCAGTGATATATGACGTTAA | |
| | | ATCCGTTCTAAGATATGCGCTTAAAATAATAG | |
| | | ACACAACCACAACACACCAAATTGAAACCTTA | |
| | | GCAAGTGCCCCATGCTTCATTTTCTTACTTGA | |
| | | CACGTTCTTTTGGTCGTATGCTTTAATGAAAC | |
| | | CCGTTACAATATCTAAAATATTACACATCAAT | |
| | | AAATACTGCATTAATGTATTCATATGTATATA | |
| | | CCTCCTTTATTAAATATTATATCATAAAAAAA | |
| | | GCACCTTTTAAAGGTGCTTATTGTCTAGTTAC | |
| | | CGTCTACAATATTTTTGATAAATGGATACCAC | |
| | | TGTTCACCACCACCATATCGAGTTGGGAGAGA | |
| | | ATCAGGCACGTTAGCACGTTCCCAAAATTGAT | |
| | | AAACATTATCAACACCCTCTCGCACACTTTCT | |
| | | GAATTGGCTAAACATTTCTTTAACGTTTCTGT | |
| | | TGTCGCCCCCTCTTTAAAAGCGTACTCCATTT | |
| | | GAGTTTGAAGTGAATACCATCCCTTTCCGTTT | |
| | | TCCCTACCATAGTTAGCTAGTCCCTCACCTCG | |
| | | TGGACCAGTCCATTGGCCTAAACCTAGTCCAC | |
| | | AATAGTGACGTGCGTCACTACCTTTATAGGCT | |
| | | TCCTCGTTTAAAGGTATTGCATAAAGAGAAAG | |
| | | AAATGATGACCATGAACCAAACAGTGTCTCAA | |
| | | CTGTTGGGTCGTTTCTCCAGTCCTCACCACCA | |
| | | TCACGCCAGTAAGTTTTCGCTTCAAAAGTGAA | |
| | | CGGGTCAATACCGCTTTCACCAACAAAGTTTC | |
| | | CAACAAGACCAGCAATACCATAAGCATTGGCA | |
| | | TTACCACAATTGGCTAGACACATTTTAGTGAT | |
| | | ATATCTTACCTTGTCTTCATCACTCATATTGT | |
| | | CTTCATCAATTGATGGGTTAGAAGTTGGTGCC | |
| | | CCTTGTGGTGTATCTGTAGCAACTGAACCTTG | |
| | | TGATGATTTTAGTAAACTTTTTAAAGCTTTTT | |
| | | CAAGCTCGTTTTTAATCTCGTCAAGCGCTTCA | |
| | | TCTGATAAACGAACTCTCCACAAATTCATGTT | |
| | | TCTAGTAAGCTTAACAACCTTATTAAACATGT | |
| | | ACTTATCTGAAGCTGTATAGACGTTTTGATTA | |
| | | AACATATCTTTGATTTTATTCATAAATTCGTT | |
| | | TAATGTTTTCATTAAATCTATTTTAATATCAG | |
| | | TGCCCGGTTTACCAGAGCCAGGTTGTGGTGAT | |
| | | GATTGTGTTGAACCTGATGGTGATGACCCACC | |
| | | CCACCCGTCATCATCTGAAACAACAGCACCTA | |
| | | GGAAGCCAGCCCAGCCACCTGCACATAGTTTA | |
| | | CCCATGTTTGCATAGTGTGCTGGTTTGTTAGG | |
| | | CGTTGCTCCGTTTTGTTCAATGGTATCATAAT | |
| | | CCTCTCCCCAATTTCCAGCTTTAATAGAAATG | |
| | | TGGCCAGCCCAACTATAAGACCCAGTTGTCTC | |
| | | CCAACAGCAGATAGCACCCTTTGGAAGCGAGT | |
| | | TGTAAATTCTAGCCGAGTCAGCGTCATCTCTA | |
| | | GGATTTCCTGCTACAGTGTGCCAACCAGGCGT | |
| | | TGACAATGCACCGCTAGCATAATCAATATAAA | |
| | | AGTTTTTTGCTGCGTCGCCACGAACATATGTT | |
| | | GTTAACCCAGCTCCTAAATCACGATTAAGCCC | |
| | | TGAGATAAGTGCGATACATTGATACCCAGCGC | |
| | | TGGGAATATCAATATTACCCATGCCAGAATCT | |
| | | GCCCAAGTTTTTGCTAAATCATAAAAATCTTT | |
| | | ACTTGTTACCATATTTTCTCCTTTTCTAAGCA | |
| | | ATCACATTATTTTCACTTAATTCACTTCGTGA | |
| | | TGCCAAATCAGCATAATTGTGCCACAATCTCA | |
| | | CACCACCTTCAAATAGTGTTTTGAGTTGGTCG | |
| | | AAAAGTTCTCGGTCAATATCGTCAATCCAATA | |
| | | ATTTCCCTTGAACTGTAACCAGTTAGCCTTAG | |
| | | ACATTGAATTAATGTTATAAATTTGATTATCG | |
| | | TTGTTCATTGCTTCGTGTCCAAAAGCACCATA | |
| | | ATATCGGCGCAAGCTTGTTAACTCTTCATAAT | |
| | | TAATAGTTGATACTTTCAACCAAATTCCATAG | |
| | | TCACCAGTTTTGTCAAGCACACTGTTATTATA | |
| | | GCCCCCCTCTGAAACTGTAGGTGGTGAAATCT | |
| | | TCCATTGGTTCATTTGTGCCTTTTGGCTTCGG | |
| | | TAATACTCATATTCATCAGCGAACAATCCAGC | |
| | | ACCTTTAGCGGGAGCACTAGCTAGTCCACCAG | |
| | | CAAAAACATTTGAGTAAACTGAAACAGCGTTA | |
| | | AATAGTCTGTCTTTTACTGAATTATTAGGATT | |
| | | TGTAATAGCGTTTATACGTCCAGAGATTGTCT | |
| | | TAGAATTTTCTAGCTGTCTTGAATAAGCTGTG | |
| | | TTAGCTTTGTTAAGCGTATAGTTATTAATCAA | |
| | | AACTGGTACATTATCAAACGTGTTAATTGTCA | |
| | | TCTGGTTATCACGGTAAAAACCATGTCTTGAA | |
| | | TCACTTGTCTCACGTTGTCCATATTCAAGCGA | |
| | | ATATATATCAATCTCATTAAAACCACCTAAAA | |
| | | CAGATGTAGCGTATAACGTGTTACCATCTTTA | |
| | | ATTTTACCAGTTTCAAAATTTAACTGGTTACC | |
| | | ACGATAGTCAGTTAGGTAGCAATTAATGACTT | |
| | | GGTCTCGTACCAAATAATCTTCAAAGTCTTTT | |
| | | AATCCTAATACCTCTTTAATTTTTGATTTGGT | |
| | | TAAAGTAAAAGGAAGCGTTAATATATTAGAGC | |
| | | TATTGTTACCATCTAACACATATAAGTGTGTT | |
| | | CCTTTACATTCAACCGTGTTTAATTGTGATAA | |
| | | GTCAAAAAACATTGATGGCACTTTAACAATTG | |
| | | TTTTAAAATTTTGAGTTATCCAAGGATAATCA | |
| | | GCTAACGTTGTTAGTAAGCTGTCTAAATCTGT | |
| | | GCTTGATACAAGGTATAGACTTACAGCTGACG | |
| | | TAATACCGTCATAAGTGCCACCAATCGATGTT | |
| | | TGCATTTTTGGTTTGTCTTCTGTACCAAAATC | |
| | | ACCATTTAATTTAACACTTGATTGAATGATAT | |
| | | ACATGAAGTCATTGAACTGTACACCGTCGGTG | |
| | | TTGTTATTGGTGAATACTGATGGGTCAATGAA | |
| | | CATCATTGTTGATGTTGGTAAAATGTCATTAC | |
| | | ATGTTCTTAAATACTCCTCTCGTCCTCTTAAG | |
| | | CGTTGCAAAGGCAGGTGCTGTCTGATAACTTC | |
| | | AACGTTTTGAAGATTTTCCAGTACGTTTCCTT | |
| | | GGGTGTATGTCATGACAACATCAATAACAAGG | |
| | | TCTAATTGTGTTGTTCTATCATTTAGATAAGT | |
| | | AGTTTTAACAATAAAAGCATAGTACGCTTTAC | |
| | | CGTCCCAACCATCAATGAATCGACAATAGTTA | |
| | | AAACCTTGTAAACTTTCCATATCCATAGGAAC | |
| | | TTTAACAGTTCCTCGGTCATAGCGATAATTAA | |
| | | ACCGATAAGGGAACGTTATGGTGTTGCTACCA | |
| | | GTGAAATGGGTATCAAACCAATTATCCCGCTC | |
| | | ACTATTTGAATTAAAATGCAATGTATTTTGCA | |
| | | TGTCAGTGAACATTGTATTTTTGAATAATTTG | |
| | | AATGTTGTTAGTTTCATATTTCCTCCTCTCAT | |
| | | ATATGATATTATATCACAAAAACACCCGCTTT | |
| | | TAAACGGGTGAATTTATTAGATTAAGAAAATT | |
| | | AGAACATTCCCTCTTGTTTCCATACTCCAGAT | |
| | | TGACGGCGTCTTGTTTGACCAGTGTTGTCACG | |
| | | TCCAATGTATGCAATTCGTTCGTTGCCTACAT | |
| | | CATTCCAAGTGTTTGACGTTCTAAGCCATGTT | |
| | | GATTGTTGAAGTAATTGAACCGCTTTCCATTC | |
| | | GTTTGATTTTCTAAGACCGTTCGGTTGATACA | |
| | | TTGGGATAACGTTAGTAACTTTACCACCTACA | |
| | | TATACAGCACATTCATCTGAAACAACTGCGCT | |
| | | TGCTAGCCATTTGAACGCTCTAATTTCAGGTA | |
| | | TTGTTATGGTACCGTTTGGTGGTACAACATAG | |
| | | TGTCGTCTTGGTTGTGTTGCTACGTTAATCCA | |
| | | CCCGCTATCAAACGGCAATCCAATATCGCTTT | |
| | | TTAAGTTCCACACACGTTGTAAGTTACCATTA | |
| | | TCGAGGATTTTATCAATGTTATAACCTTTAGC | |
| | | ACTTGTTGAATGTGTTGTGTAAGCACGTACCG | |
| | | CTCGAATACCTCCGTAATCGAATGACACAGAC | |
| | | AAGTCATCATGCACTGTTACATTAGCAAGGTC | |
| | | AAGTTTTGCTTCTATACGATATTTAGAAACTA | |
| | | ATGTACCAGCAGGATAGTAGTTGTTATATACA | |
| | | ATGTATATTGTGTCATTTGAGGAATGTGTATT | |
| | | ATCCAAGTTTGAACCTTGAATAATGTTTGGCC | |
| | | TCATATTGAAACTAATTTCACCCATGAATTAC | |
| | | CCCCTTGCTCCGATTGTTGCATCGTTTTCATT | |
| | | TTGACCGCTTGTTGTGCGAATGAATGTGTTAC | |
| | | CGTCTGTTGTACCAGTAAACAAATTAATGTTA | |
| | | CCGCTAGCGATATGACGGTTTGCTGTCATGCT | |
| | | ACCTGCTAGAACATCATCACCAGAAGTCCAAG | |
| | | CGCCTGAACCTTTCAAGTCTGTTAAAACTTTA | |
| | | GTAAGTGCTGTTTCTAAAACAGTGATGCGATT | |
| | | TGATAAAGCGGTGTCAGCGTTCTCTCGTGCTG | |
| | | ATGTTTCGTTGTCAATGCGTACACCTAAAGCG | |
| | | GTATCTGCGTTTTGGCGTGAGCTTGTTTCATT | |
| | | GTCAATGCGTACACCTAAAGCTGTATCGGCGT | |
| | | TTTGTCGTGTTGTTGTTTCGTTGTCAATGCGA | |
| | | ACGCCTAAAGCTTCATCACCTGCAATGCGTGC | |
| | | GTTACGTTCAGCGGTAATTTTTCGGTCTAATT | |
| | | CTTCATCAGCCGCGATACGGTCAAGAATTTCT | |
| | | TGATTAATTTTTGCTAGAAGTTTTTCCAGGAG | |
| | | TGGAAGATAGTTAGGTGCAAATAGTCCAGTTG | |
| | | GTAAACATTCAATAGCGTTCTCAATATTATAG | |
| | | CTTTGACCGTCAAATTCAACTGCTTTTTGAAT | |
| | | TGTTGATAAAATTACTTCAGCTTTCAAAATAA | |
| | | TTTCATCGTGCCACGTATGACAAGCATTGCCT | |
| | | TCATCTATCCAATCATTAATTTTAGTTAAATC | |
| | | AATGCAATTAGTATCTTCAACTTGTACGTTAC | |
| | | GACGTGCGACACGGTTTAACAACTCAACAATT | |
| | | GACTTGATAAGCTTATTAAAGTTTGATAGATA | |
| | | ATCATAATATGATGGTGCGTTGGTGTTATAGT | |
| | | CTCGGCGGTCATCGTACCATGGTTGCCAGTGC | |
| | | CCTTCTAAAGAAAAAGGATAGTGTGGGGCGAA | |
| | | GTATGGTTTGTTTTCTTCAAAGTCGATACTGT | |
| | | TAATATTTTCCATTTTTTTTTCCTTTCTACAT | |
| | | AATTTGTGAGAACAACATTCTGTCTAGGTCGT | |
| | | TAAATAGGTCATTATGAAAAGTGTATAATTCT | |
| | | CTCATGCGACCGACTTCAAAAGTTTCTTGAAT | |
| | | TCCAAAATCGTCTGAAGTCGTTTCATTAGAAT | |
| | | TATTTGACCTAGATTTTGAGTGTGCTGTTGTG | |
| | | TCTGCATAGTCATAAGTTTCTTTATCGAGCGA | |
| | | TAAGTCAGTGTTATCTTGTGGTAATGTGACGG | |
| | | CTAGGTTGTTATCTCTAGTTGTTACAGTACCA | |
| | | GTAGAATTAGAAGTAGAGTTGCCATAAGTATA | |
| | | TACATTATTATTAATGTATTTTTTACCATTAA | |
| | | CATAATAGTCATTAATCACGTCTTTAATGCCA | |
| | | CGAATAAAGCTAACTAACCGCCAGTTAAACGT | |
| | | TTCATAGGTTTGAAACTTGATGGTGCGTGTTA | |
| | | AGAACTTAGCTAGGAACTCTGTTTCAAATTCT | |
| | | TCTCGGACTTTCTCGTCTAAAAAATCAAGACC | |
| | | ATAAAAAATAGTGTTACGGCAAACGGTTTTAA | |
| | | CTTCCTCATCATACTGAAGTACTTTGTGGGTA | |
| | | AATTGTAGTTCAGGATTATAATAAACAATCTG | |
| | | GTTAGAACGTAAAAAATCGCTGTAAATATTGT | |
| | | TATAGGTTGTGTAAATAATGTCATACAATCTT | |
| | | GTAGTTGTCTTCATTAAATACCTCCATCTAAA | |
| | | ATTTGTACTTTTTCAAGACTTGAAAGTTCTGA | |
| | | AGCGATTTCATCTCTAAAGCTTGCTTTAAGCC | |
| | | TAGTTTTGAATTTATCGTTATAGTGTTTTAGC | |
| | | TTTTCATTTCTAGCTTGTAAGTAAATACCTGC | |
| | | GTTTGCTTTTTGATAAGCTTTATTGCTTTCGG | |
| | | CTTCTGATTTTGAAACACCGCTCTCTTTATCA | |
| | | ACAGCTAGAGAAGTAAGTCCTAAAATGTTGTT | |
| | | TAATTCAGCAATGTTGTTTTGGTATTCTCGTT | |
| | | TTAGTTCAGGTAAAGCACTGATAAGTCCTTGA | |
| | | CCCTCGTTAATTGAAAGAATACTTTCGTCAGG | |
| | | GTCAAAGAATTTTGTTGTTTTAATAAATGGTG | |
| | | CGCCGTTGTATAAATCTTGGGTGATTTGCTCA | |
| | | ACGTCCTCATCGTTCGCCTCGCCTCGGATAAC | |
| | | TGTTGAAATTTTAGCTTGCATGTAAATGCTAA | |
| | | AGCGTGACATAGCAATTTCAGCAATGCGTTCG | |
| | | CTGTATGTTTCGATGATATTAAAATCGTTAGT | |
| | | TAGCTGAATTGGTTTGTTCCAAAGAACAACGA | |
| | | AGTTCCCAGATTCATAGCCATCGTGGTAAGTG | |
| | | ATTTCCTTGTAATATTTTTCTTTGATTTTGTT | |
| | | AGAGATAAAAAAGTTAATGTCATTACCAGTCA | |
| | | AAGGTCTAGTACCGTAGGTTTGAATATTGGAA | |
| | | ATTGTATTTGATTGGTTAACAGTACCTAAAAT | |
| | | CATAGTTCCTCTTTTAGTTTGACCAATAGCAA | |
| | | CACCGTAGCCTTGACGTAGCCAGACTTCTAAT | |
| | | TGAATAGGGTTAATTAAAACATTTTCATCATT | |
| | | GATACCGTCATATCCAATAACTGCCGGTAAAA | |
| | | ATTCAATGTAACGGTTTCTAAAGAAATTGAAA | |
| | | AAGTTATTTCTTTGTGATACAACCCTCATTTT | |
| | | AACCTTTTCGGATAAGGTTTGCTCAATATTTG | |
| | | AATAGAAGTGTGTCATTTTTCTCCTTTCCGTG | |
| | | AGTTGCGTTAGGACTTTTAGTCCTTACGAACC | |
| | | GCTCTATTTAAAAATAAAAGCAGGGACAAGCC | |
| | | CTGCATTATTAGGCTGTTTCGCCTTTGATGAC | |
| | | AACCTTGTTATAGAATGGTGAAATTGCTTTAA | |
| | | ATGAATAGTAGTGAATCCAGTAAGTGACTTCA | |
| | | TCAAATTCACCGTTGTAGAATGGTTGTTTCAA | |
| | | CATTCCTTTTGTAAAGCGTTTGTAACGAATTG | |
| | | CTCGTGCGTCTAGAACCATTGCAAACAAGTCT | |
| | | GATTCTGGTTTAATTTCTTCAAGGTCTTTTGT | |
| | | AATCACGTCTGAAACATCATAAGTGAATACAT | |
| | | AGCCTTTAGGAATAATGTCACCAACTTGAACT | |
| | | TGATAGTCGCCGAACAATTGAAGTTTGTTAAG | |
| | | GATAGTCTGTGTTACAGTAATATCTGCTGTTG | |
| | | TGCGGTAAGCCCCACCCAAGTCATCGAATGAA | |
| | | ATAATGATTTTTGATAGGTCAATTCCCTCAGC | |
| | | GTGGAATGTGTTAGCTAGGAACGTGTTTAATA | |
| | | GGTAGCGTTTAACTTTGTCAGTGGTGATGATA | |
| | | ATCATATCTGAAAGTTTAGTGTTAGTAGTGAA | |
| | | ACGACCTAGAGCGCCGCCTGAAGCCGTAGAAG | |
| | | CTTCGTTGTGTTTAGCGCTGTTGTTTTGAAGG | |
| | | TTAAGGATTGCTTCTGAAAGTGTTTCAAACAA | |
| | | TTCTTCCATGGTTGTAACGGTGCGAGTGTCGG | |
| | | CAACGTGGTGAAGACCATAATCAACCAGCATT | |
| | | GCTTTTACTTCAGATTCTTCGGCAACGTTGAT | |
| | | ATCAGTGATTTTCTTGCGGTATACTGATACAG | |
| | | CGTAAGTGATAGCGTCACCGATTGTTAAAAAG | |
| | | TTTAGGCGAGTATCGTTGTTGTTAAGTGTGAA | |
| | | TTTGAGTTTCTTAAGAATACCTTGTCCGTAAA | |
| | | GTTTTGTAGCCATTTTTGGATAATTACGTTTT | |
| | | AGCATAAGTTCGGCATTCTTAGATAGGTCAAG | |
| | | TTCAATTGGTACACTGTCCAAAATAACATATT | |
| | | CTTCAGAGTATTGACCGATGAAGTCAACCTCT | |
| | | TTAGCTAGCCAGTTAAAACGGTTTCCAAGAAT | |
| | | TGACTCAATGATAAGTGTTTCGTTAAGTTTTG | |
| | | GAAAAAGATATTTGTTAACGTATGTTTCAAAT | |
| | | TCTTTTCCTTGATTGTTCCAGTTAGTTCCAAA | |
| | | CGTCCAAGCGTGACCTGTTTCGGTGTTGTGCG | |
| | | TGATAATAGAAGCTTGAACTGCTTGTGCAAGT | |
| | | GCGTCAGCCATTAGTTAGTACCTCCAATATTC | |
| | | ATAATTTTTTGTTCGTAAGCGTCACGTCCTTG | |
| | | TGGTGCGATTGACATTGATTCATTCAATTCAT | |
| | | TGGCTTTTGATAGTTTTTCCATGCCAAAATCA | |
| | | CCGCCAGAGTTAAATGTCTTTGCTGTTCCTTC | |
| | | GATTGGTTCCATAGTGTTTCCTTTCTATTCTG | |
| | | CTAGAAGTTCTTCTAGGTATTTTTGAGATTCA | |
| | | AGTTCTGATACTTCATTTTGAAGTTTTTGAGC | |
| | | GTCTTCAAGTGCGTGTTGATATGTGATAAAGT | |
| | | GTTGATACATGAGTTTATCAAGTTTAGCAATT | |
| | | TTATCAGTGTCTTTTTCATCAACGATTGCTTT | |
| | | AGCTTTCACCACTTTAATATATTCACTGTAAG | |
| | | CATTTGTTTGAGTAATTTGAGCTTGTGATAGC | |
| | | TGTTCACGTTTTTGTGAGAGCTGTTCATCGAT | |
| | | AGAAGTAGAAGTAGAAGTTGTTTGTTCTGCCA | |
| | | TTTTTTAATCCTTTCTATTAAGAGTTGAGAAG | |
| | | TTTTTCAATTTCATCTACAGATTCAACTTCAG | |
| | | GTTCTTTGTCTTTTGTTTTGTCTTCGTCTTTT | |
| | | GGTTTGTCTTCATCTTTTGTTTTTTCGCTAGA | |
| | | AGATTGTTCGATAACAGAAAGACGTGACATGA | |
| | | TTTCATCTAGAGTTGAATTTAAATCGTCTTTT | |
| | | GTTTTGATTTCCAATTTTAAAACTCCTTTCTA | |
| | | AAACATAAAATGTAGCACAATATTATTAGTCA | |
| | | TACTGTCATGTGTTACTTAATTATGTTTTAAT | |
| | | ATTGTACTAATTTTTATAATCTAATTATAATA | |
| | | AAAAAAGAAGCCATTGTCAATAGGTTCATTGT | |
| | | TTGGTGATAGTAAGTGTATACTTTTGGCATTT | |
| | | CATACATATGTAAGTGTGTTTGCCTTTTCTGT | |
| | | TAAAGATTTCATATACTTTACAAATAGGACAG | |
| | | TAGTGTTGTGGAAATTCATTTGAATAATTCAA | |
| | | TTGTGTTCACCTCACTAATTGTTATATCTGAA | |
| | | ATACCTGTCTTTGCATTTAATTTTGAAAATAA | |
| | | TGAGTTTAATTGTTCGCTAGAGCATTCTTTTT | |
| | | CATTGTAATGACCAGCGTGCCAATCGTACCAT | |
| | | TTGAGTTTGTAGGTTCTTGTCATTAATACTCC | |
| | | TTATCTATTAAAATAAAATATAGTGTAATAAT | |
| | | AGCACTGATAGCAAAGAATTGGTAAACTGGTG | |
| | | AAATTGCTGTTAAGCATGACATTGTGAAAGCG | |
| | | AGACCGAATAGAGTTAGTGGTAGCATAGTTAA | |
| | | TTACCTTTCTTATTATAAGAACAATAAAGTAA | |
| | | CAAAAATCATTAGTAAAACAACGATAAAAGTG | |
| | | ATGATGAATGCTAGTGCATATAGTAGAAATAA | |
| | | AGCCATTTTATTGCTCCTTATTTAATTAGTGA | |
| | | AATTTTGATATTATCAATATAACAGTGTTTGC | |
| | | CTAAATTTTTAAGGGTTATTAAACATGTGTCA | |
| | | TAGTACCTGAATTGTACTTCAATGTTTTCAAC | |
| | | ACCTTCAATAGTGATTAAGTCATATGTTAGTT | |
| | | TCCAAGATTTATTGTTCATTTTAATGCCTCGT | |
| | | CATAAATTGTTACATTGTCCGCCATAATTAGA | |
| | | TAAGCACCGTTTTCTTTTAGTAAATTAACATA | |
| | | TAATTCAAATAATTCTTCATTGCATGTTATAA | |
| | | CATATGTGTGAGTATCGCATGGTAAATTGTAA | |
| | | GTTAATGTTTTCATTGTTTTGCTCCTTTAAGT | |
| | | GCTTTTTTAATAGTCATTTCTTTGACTAGTTT | |
| | | AATTTCTTGCTTTCTTAGATTTTCTTGGATTG | |
| | | ATTTTAGATAGTTAATTGCTTCAACACTTATA | |
| | | GTCATTTCAATACATTCATCGATGTTTTTAAT | |
| | | ATCGTTTTGAATTGTTTTGAGTAGTTCTTTGT | |
| | | CTGA | |

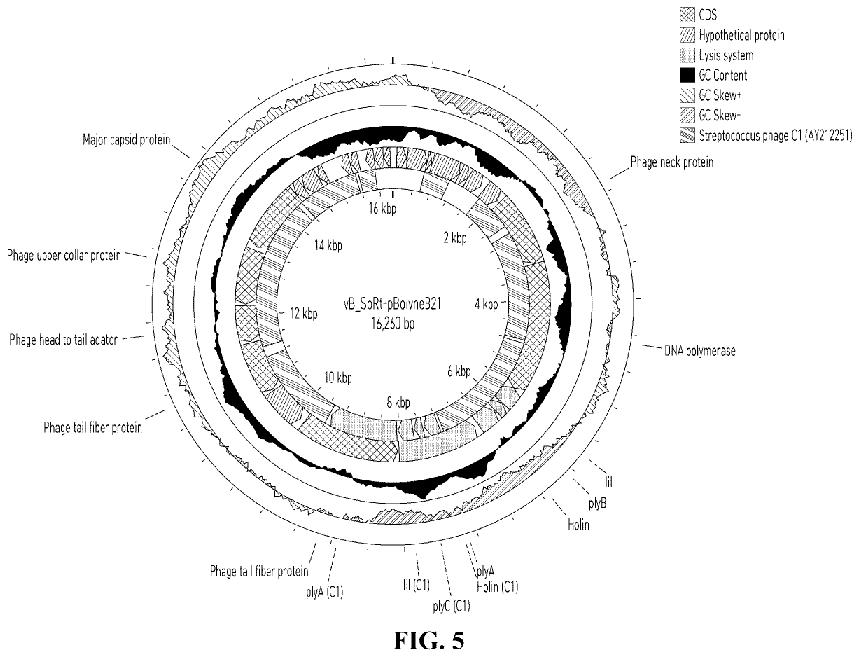

As a result of the analysis, as can be confirmed in Tables 3 to 4 and FIG. 5 below, the genome of the obtained phage vB_SbRt-pBovineB21 was identified as a double-stranded DNA consisting of 16,260 bp with a G+C content of 33.7%, and was confirmed to be composed of 27 ORFs.

TABLE 3

| | | Length | GC | | Genome |
|---|---|---|---|---|---|
| Strain | Contigs | (bp) | (%) | Coverage | Characteristics |
| vB_SbRt- | 1 | 16,260 | 33.7 | 100x | linear |
| pBovineB21 | | | | | |

TABLE 4

| Genes | | | | | Best Match | |
|---|---|---|---|---|---|---|
| ORF | | | Length | Putative function | virus | Identity |
| No. | Range | Strand | (aa) | [Conserved domain] | (E-value) | (%) |
| 1 | 47-222 | + | 57 | No match | No match | — |
| 2 | 234-602 | + | 122 | Hypothetical protein | Lactococcus | 50 |
| | | | | | phage P1046 | |
| | | | | | (2e−12) | |
| 3 | 599-688 | + | 29 | No match | No match | — |
| 4 | 685-1212 | + | 175 | Hypothetical protein | Streptococcus | 25.5 |
| | | | | | phage C1 | |
| | | | | | (0.06) | |
| 5 | 1212-1364 | + | 50 | No match | No match | — |
| 6 | 1367-1645 | + | 92 | No match | No match | — |
| 7 | 1707-2027 | + | 106 | Hypothetical protein [PF16773; | Streptococcus | 51 |
| | | | | Single-stranded DNA binding | phage C1 | |
| | | | | protein; Lactococcus phage p2] | (1e−26) | |
| 8 | 2066-3313 | + | 415 | Putative encapsidation protein | Streptococcus | 50.1 |
| | | | | [7JQP_A; Encapsidation protein; | phage C1 | |
| | | | | packaging motor; ATPase, motor | (2e−138) | |
| | | | | protein; Lactococcus phage | | |
| | | | | asccphi28] | | |
| 9 | 3332-5650 | + | 772 | DNA polymerase [PF03175; | Streptococcus | 53.3 |
| | | | | DNA_pol_B_2; DNA polymerase | phage C1 | |
| | | | | type B, organellar and viral; | (0.0) | |
| | | | | Streptococcus phage Cp-1] | | |
| 10 | 5647-5988 | − | 113 | lil [PF13392; HNH_3; HNH | Streptococcus | 50.5 |
| | | | | endonuclease; Bacteriophage | phage C1 | |
| | | | | SPO1] | (1e−21) | |
| 11 | 5957-6169 | − | 70 | No match [4F87_A; PlyCB; lysin; | No match | — |
| | | | | antimicrobial protein; | | |
| | | | | Streptococcus phage C1] | | |
| 12 | 6162-6551 | − | 129 | Holin [PF05105; Phage_holin_4_; | Streptococcus | 38.7 |
| | | | | Bacteriophage holin family; | phage C1 | |
| | | | | Streptococcus phage Cp-1] | (6e−11) | |
| 13 | 6617-8044 | − | 475 | plyCA [PF18013; | Podoviridae | 40.2 |
| | | | | Phage_lysozyme2; Phage tail | sp. (3e−11) | |
| | | | | lysozyme; Streptococcus phage | | |
| | | | | C1] | | |
| 14 | 8058-9809 | − | 583 | Phage tail protein [PF16838; | Streptococcus | 37.7 |
| | | | | Caud_tail_N; Caudoviral major | phage C1 | |
| | | | | tail protein N-terminus; | (6e−103) | |
| | | | | Streptococcus phage C1] | | |
| 15 | 9887-10585 | − | 232 | Hypothetical protein | Podoviridae | 35 |
| | | | | | sp. (8e−35) | |
| 16 | 10589-11533 | − | 314 | Tail fiber protein [6IAB_A; Tail | Podoviridae | 38.5 |
| | | | | fiber; structural protein; receptor | sp. (4e−57) | |
| | | | | binding protein; Staphylococcus | | |
| | | | | phage P68] | | |
| 17 | 11547-12206 | − | 219 | Head to tail adaptor [6Q3G_BC; | Podoviridae | 38.2 |
| | | | | Lower collar protein; structural | sp. (4e−30) | |
| | | | | protein; receptor binding protein; | | |
| | | | | Staphylococcus phage P68] | | |
| 18 | 12206-13168 | − | 320 | Putative upper collar protein | Streptococcus | 45.5 |
| | | | | [PF05352; Phage_connector; | phage C1 | |
| | | | | Phage Connector (GP10); | (6e−77) | |
| | | | | Staphylococcus phage P68] | | |
| 19 | 13257-14411 | − | 384 | Major capsid protein [6IAT_A; | Streptococcus | 63.6 |
| | | | | Major head protein; structural | phage C1 | |
| | | | | protein; Staphylococcus phage | (1e−179) | |
| | | | | P68] | | |
| 20 | 14411-14572 | − | 53 | Hypothetical protein [6IAT_E; | Enterococcus | 61.3 |
| | | | | Arstotzka protein; structural | phage | |
| | | | | protein; Staphylococcus phage | vB_EfaP_Efmus3 | |
| | | | | P68] | (7e−04) | |
| 21 | 14585-14881 | − | 98 | No match | No match | — |
| 22 | 14898-15040 | − | 46 | No match | No match | — |
| 23 | 15342-15511 | − | 55 | No match | No match | — |
| 24 | 15511-15642 | − | 43 | No match | No match | — |
| 25 | 15762-15923 | − | 53 | No match | No match | — |
| 26 | 15920-16078 | − | 52 | No match | No match | — |
| 27 | 16073-16197 | − | 40 | No match | No match | — |

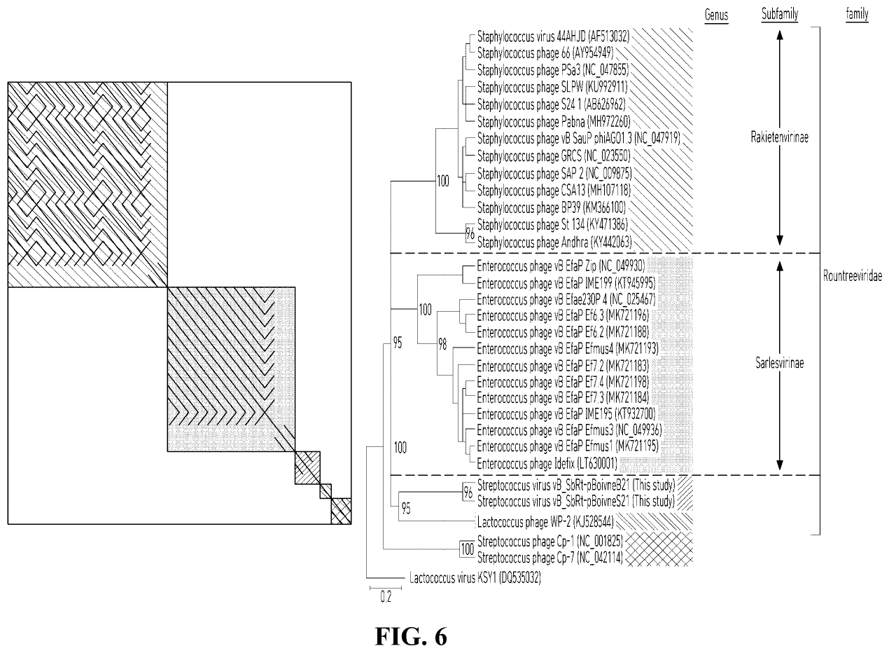

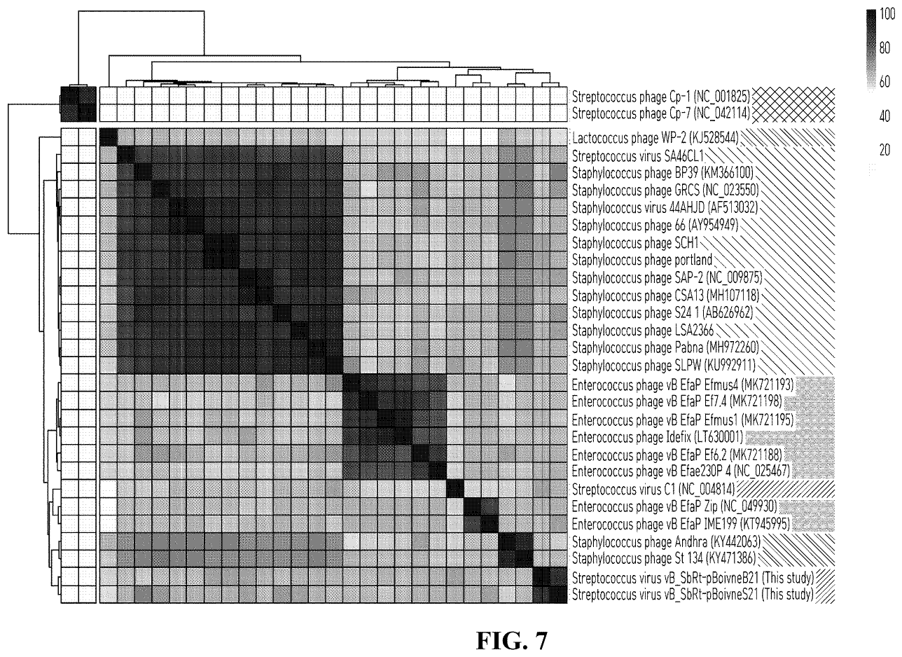

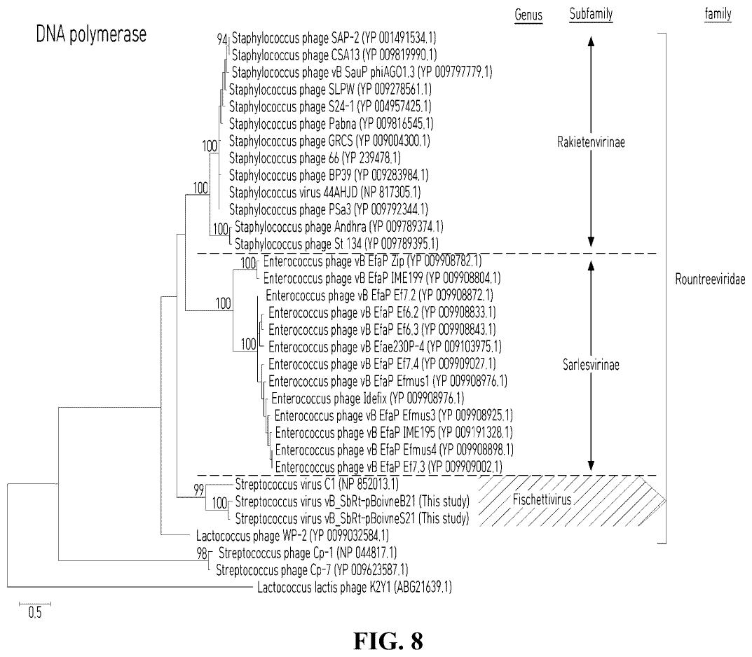

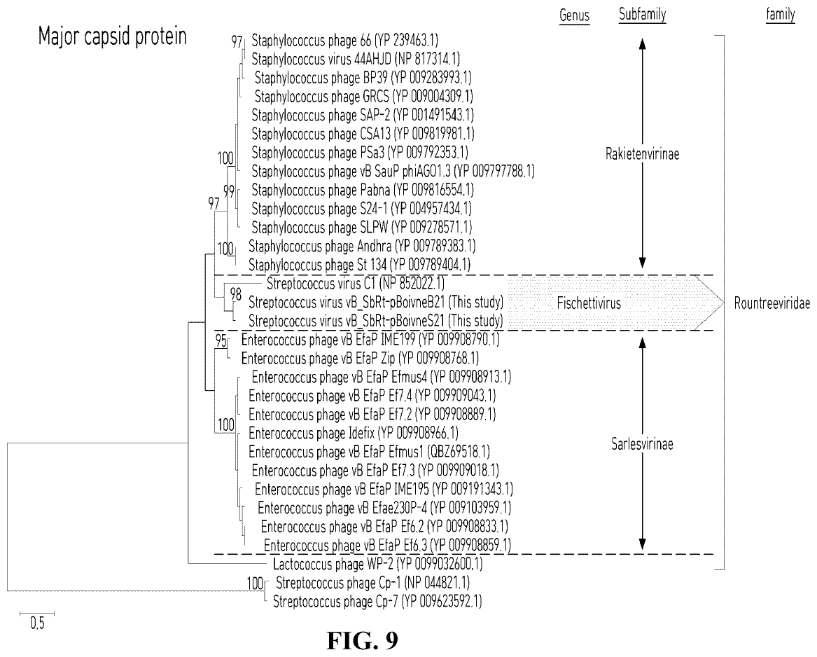

To compare and analyze the genetic characteristics of the phage vB_SbRt-pBovineB21 with other phages, phylogenetic tree analysis was conducted using the VICTOR (Virus Classification and Tree Building Online Resource), R package heatmap, Dot plot, and MEGA X, based on whole-genome, ANI (Average Nucleotide Identity) values, and two major proteins of the phage (Major capsid protein, DNA polymerase). As can be confirmed in FIGS. 6 to 9, the bacteriophage vB_SbRt-pBovineB21 was identified as belonging to the family Rountreeviridae and the genus Fischettivirus, and was confirmed to be grouped with phage C1, which is a representative phage of the genus Fischettivirus.

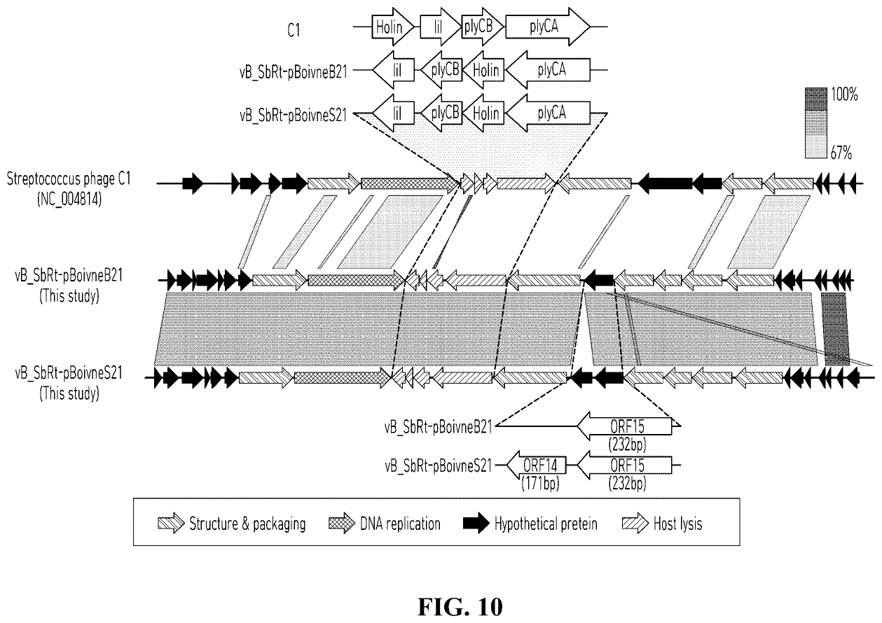

As a result of comparison of major proteins of phage C1, which shows genomic homology with phage vB_SbRt-pBovineB21, using the EasyFig tool, as can be confirmed in FIG. 10, low homology was observed in most proteins. Among them, it was confirmed that there were differences in the arrangement of four protein sequences of the lysis system related to the destruction of host bacterial cell walls. As such, the genome of the phage vB_SbRt-pBovineB21 is identified as a novel phage distinguishable from other phages, and the inventors of the present invention deposited the phage vB_SbRt-pBovineB21 at the Korean Culture Center of Microorganisms and were assigned the deposit number KCCM 13031P.

### Example 5: Verification of Biofilm Control Ability of Bacteriophage vB_SbRt-pBovineB21

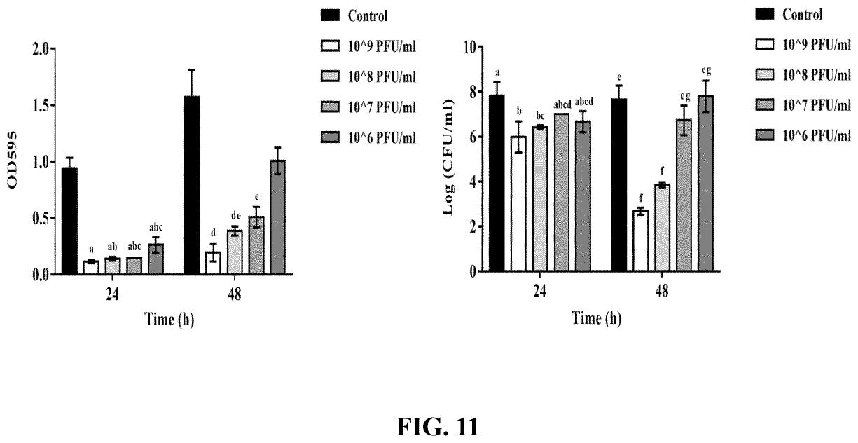

The biofilm inhibition ability of the bacteriophage vB_SbRt-pBovineB21 against host bacteria was confirmed. First, in order to verify the biofilm inhibition ability, S. ruminicola KCTC 43306 and phage at various concentrations were inoculated into TSB supplemented with 1% sucrose and 1% calcium chloride in a 96-well polystyrene plate, and were statically cultured for 24 hours and 48 hours for biofilm formation. Thereafter, each well was stained with 0.1% crystal violet, dissolved with 95% ethanol, and the value at 570 nm was measured using a plate reader. As can be confirmed in FIG. 11, it was confirmed that the phage vB_SbRt-pBovineB21 effectively inhibited the biofilm of host bacteria at all concentrations (10⁶, 10⁷, 10⁸, and 10⁹ PFU/mL) tested.

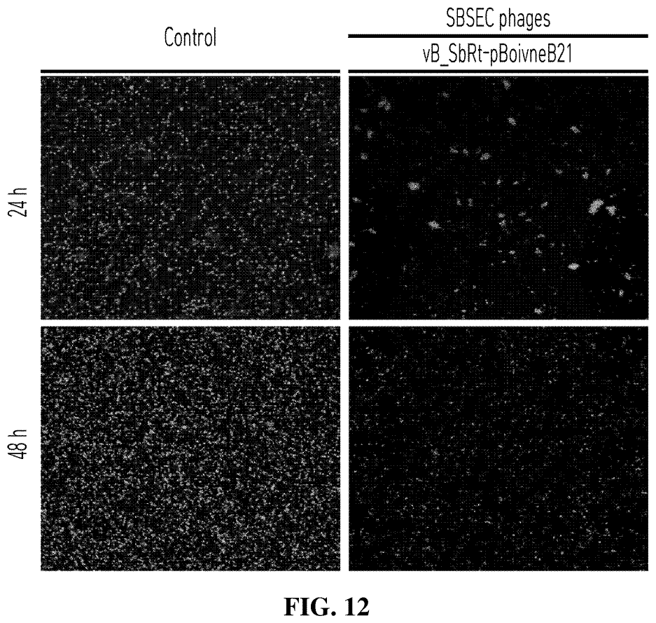

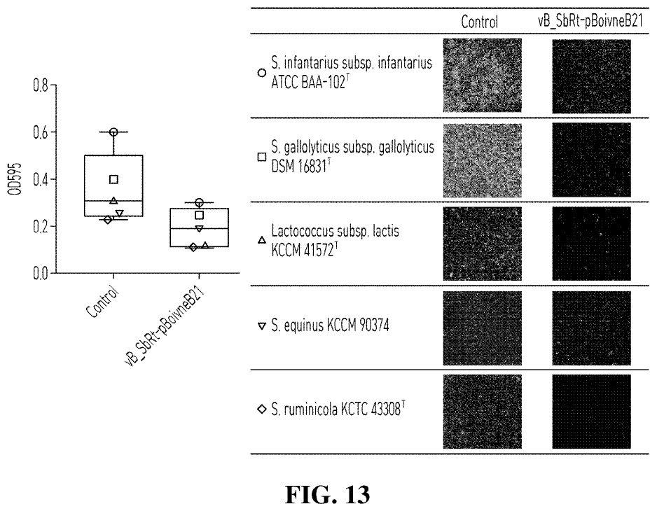

In addition, as a result of confirming the formed biofilm using confocal laser scanning microscopy (CLSM), as can be confirmed in FIG. 12, it was confirmed that the biofilm of host bacteria was effectively inhibited, similarly to the result of staining with the aforementioned crystal violet. As a result of analyzing the biofilm inhibition ability of SBSEC bacteria and lactic acid-producing bacteria used in the host range analysis of the phage vB_SbRt-pBovineB21, as can be confirmed in FIG. 13, the phage vB_SbRt-pBovineB21 was shown to effectively control biofilm compared to the control group.

### INDUSTRIAL APPLICABILITY

The present invention relates to a bacteriophage vB_SbRt-pBovineB21 having lytic activity and biofilm control activity against Streptococcus bovis/Streptococcus equinus complex (SBSEC) and lactic acid-producing bacteria, and more specifically, to a bacteriophage having bactericidal activity and biofilm control ability against bacteria that cause rumen acidosis in ruminants.

1. A bacteriophage vB_SbRt-pBovineB21 having lytic activity and biofilm control activity against Streptococcus bovis/Streptococcus equinus complex (SBSEC) and lactic acid-producing bacteria.

2. The bacteriophage vB_SbRt-pBovineB21 according to claim 1, wherein the bacteriophage comprises the nucleotide sequence of SEQ ID NO: 1.

3. The bacteriophage vB_SbRt-pBovineB21 according to claim 1, wherein the bacteriophage has been deposited under deposit number KCCM 13031P.

4. The bacteriophage vB_SbRt-pBovineB21 according to claim 1, wherein the SBSEC comprises one or more selected from the group consisting of Streptococcus ruminicola, Streptococcus equinus, Streptococcus lutetiensis, Streptococcus bovis, Streptococcus infantarius subsp. infantarius, Streptococcus gallolyticus subsp. gallolyticus, Streptococcus gallolyticus subsp. macedonicus, Streptococcus gallolyticus subsp. pasteurianus, and Streptococcus alactolyticus.

5. The bacteriophage vB_SbRt-pBovineB21 according to claim 1, wherein the lactic acid-producing bacteria are Lactobacillus strains and Lactococcus strains.

6-10. (canceled)

11. A method for treatment or prevention of rumen acidosis in ruminants, comprising the step of administering the bacteriophage vB_SbRt-pBovineB21 to a ruminant.

12. A method of lysis of Streptococcus bovis/Streptococcus equinus complex (SBSEC) and lactic acid-producing bacteria, comprising a step of treating with the bacteriophage vB_SbRt-pBovineB21.


SEQUENCE LISTING (1 sequence)

SEQ ID NO: 1 - DNA, 16260 bp, unidentified
  source 1..16260: mol_type = other DNA
attaatgtatactctgtagaacttctagacactaaagagcatgtatatatggagtacgctttattagctgacaacatgaaatcagctattgaatgtgctaaagaaaaagcttacaacatgattagtgaaaacaaaattgttattcgttatgtttcagtacaagacggcaacgaacaatatgtatacaatgtatttactaaaaaacttgaaaaatggtattaaggagcaacaacatgaacaaacaagaattaattaaagatattgaatttaaaaaaatagacgatgtgtttactgatttatcaaatggatataatttaggattatccacagcaattgaagttataaaagaatttaacgacactgaacaagagttaaccgttgattttaaacaacaacagccttttgtgtcttccactccacctcactaccagggcacaatccaacctattgacctcatcaacgctcaagaccttaactttaaccttggaaacgttgttaaatatgtctgtcgtgctggtaaaaaacaaggcgagaacatcttaactgacctagaaaaagcaaaagattacatcaactttgaaattgaaagagtaaaacaatgaatgaaataaaactaaagttattagttaacttattactattatttttatggtcaatatattttatatttaaaggtaaaaccaaatgaacaaacgtcaacgcaaaaaactagcaaaaaaacgagctatgaaaacaggtagttatactcccagaaatacttttcgctctactatgtctatcaaatcaacattattcgactctaatcaaccaaaaaatgaaaaaatgatatatctagctgaaagaaacttaaacagactagcatttgttatcacaaaaccaaatatcgttaatctaacaaacctaacagaagatgacgttaaatatattcaatcaccaaaatttactcaaacagtatctaacatacaagaatactttaactatgaccgtgctagataccgagacccagaacgagttgataacattacacgtcacgtcatgaccaaatacttctatcaacgtgatattgaaagcacacgtgcactagatattgctatcaaacgactaacaaaacaactagaaaaaataactaaaaaatactcaaaaggaaaagacagacgtgcaacaaataatccgtttgttatcgtctattaccaagacttggagctaagataatgaaatatttaattacatatcacacaaaactagacaacggctatattgatgaacgtcaaatcatttgtaagaacaaatacaaagctaaaagaatttatgacaagttagcgggaaaaaagcgaactatcaatattgaattagtcgaggtataaacatgaccactactacttccacttaccgtgtgtcaatcaactataaaagcaaagttattttactttcaaaacctttgaaagaatgcaaatactttcacaattatgccaattatattgacactacttataaattaaataatcaagttgtaaatgtacactttgatactaaaaatcaccgtgtgaaccattacacaccgaattataaaacagctaaacttttatttgaaaaacttaaaaacacgaaaaaagattacttttttttcaattctttagataattagcttgacaaacaaaaaacaactattatataattaacttacaaacatgaaaggagccaaacacatggcattacaatttaacacaatcggatttatcaacttcaacaaagattacaacaaagttttgaagtcaggagctatcacagtttcattcacttctactattaaaaataaagacggtgaatatgaaaaacaatatttcaatggtcttatccctgctaaactagcacaacacgttaaaccatacctaaataaagaacttgtaaaaattcatggtattgcttctcctggtcaaaaaggatatatcaactttacaattctagaagttgaacaatataaaaaagaaacaaaatcagacgtgaatgacaatgacttaccattctaaaggaggtgacgggaggtgaatagcctcccttttattttatggctaaaaagaaaacattaccaaaaacactaaaacaaaaagctgtatcactttttgcaaccgaaacagaagaatatacttattatttaaacgaataccgctcaaaatacctaccacctgaatttaaccaattagaactgttagatgaactttgtaacgaagaactcgaccattatatgagcgtcaccacacgtggtgatggtaaatcatttaactatatttcagcggttggctacctctgctatcaccttaacatgggctgtactttacttgtccgccactttactttacaagacaaaatgagagaacttgttgaagacatcttacaaactatcggctggtgtaatttctctacagattaccactaccgctctacttcagattatttaatcatttctattggtgacaaagacgtttttcttatcactgacataaacaacgctagcgacttgaaacagtcttctgctgttttgaaaaaatttcctatcatgctatacgatgaatttctaacattaccagatgattattgtaaaaacgagtacgaaaaaattcgcaccatttacaaatctattgaccgtgttaaaaaccgtccgtatatcaaaacacctaaaatgatttatctagctaacccagttaactttgatagtccactactaccatcactaaaaatctataacaaattacaaacacaagaaattaacacaattaaacaatataacaacgtcttgttagaactacgccgaaacgattcacgcaatgacggaaagaacctacgtgcctttcctgatggtgatgacgctgacgttactggtgaatttcaattctcaaatcacaagctggtttccgaagaaacctattttaatatctataaccacgcaaaatcagtaaaagttaaattagacgataaattaatgttgcatatcattgctaaaaatgataatatagtattatcaatagaaagaacagataacactgaagaatattgtataaatttagttgatgaaacagaaaaaagcaagtatttaaacgataaatactacaaacaaagcttcatcaagaaacatgaaaaaggtatgtttctttacaaggacagcttttcaaaatcttatatggaacgtgacacaatgcttatgcaactaaacttgtttaagcttatcccgacagctaaagaaattacaactgaagaaacatatatcaaaatcaaggaaaacaatttccttaaatctctagctcaaaaatatgaataataaaggagtcttaatatcatgcacaatcaaaatgaactacttgactttttaaaaaagtttaaaggaaagaaagtctcattatatgctgatattgagacttttacttgtaacaaaattgaaggcagtgaacacccaacaaaataccattcttttacatactcactagccattgcctattttaatgatagcgactttccaaaagtagcagtatttaataatttttatgatttttttgaaaaagtaaaagaaagaaaaatcagaaaatcgctttctttcgattttgtctttcacaatggcgaaaaattcgataaccacttttttatcgaagaaatgcaagcttactatggtttaccagtttacactgaatacaacaaaaacgcaaataacctagctaacgaacacgctagaaaaatgtctaccattgatactgaagaaaaaaaacacggtcttgttttggagagtcgtgtaaaatcttctaacaacgtatctgttaaagcgttcgttcatggcagacgtgttgaatttattgactcctttaaaaaaatgaacacatctatcgctgtactcggtaaaatgttacttaacaataatttaatcactgaagaatatttgaaaaccgattttgactatcaatgttttgataaagatgaagacattgaaagagagctagttaaaacatatgttaagcgttgttttgaaagtcttaatgaaaaacaaatgatttatatcagaaatgacgttattattttagcattaggagttaaacattataaaaccttattctatggctttgacttttctaaaataacctttactcaaaatatcaaagaggaatactcaaaatataacaaactagctgaatttcaactattaaaaacagacggtcgcttctctcatttaaaattaaacgattatcaaatatgtggcatgtctggttttgactatttccgttcatattacaaaggcggtttaaacctatacaatgataaatacataggaaaaatattaaatagagacggtttttctattgatttaaacagttcttatcctacagtcatgtataaggagaagttaccaacatatttaatagctttaaacgaaaaaaaatcaattgccaattttgactataacaacaataatattatgtcgttctttacaatgacaattgaaaacgctaataagtatatactttctaaaatagaaagtaaagtcttaagaaatgcaatcgttaaatactacaattcaaaaaatggtcttgtttactataacaccgttctattaagactactatctaaaattacaaagaaagactttaagtcacttccagttgaaagcactgctacatttcaatgtgaatactttggagcacgtgacgtgattgcccgtaactattttattaaatcacaagggaaaatgaaaaacaaactggcttgtgagattgatacaatcgaccccctaaacatagaaatgactaatgaaccaaaaccagctaaatataattttagtgatgaaatggtacaaggttcaaaagtccttttaaatgggatttacggtgttccagctttacgtattcattttgatatttttaaacgtgtcggtaacgactttgaaaacgttaaaaacggctttaccaataaagaaagaaatattgttttttctgctggtgttactgctttcgcttttcataatctactatcaccacttcagtatttaacacccaaagaaattgatgaatatttctggtatgctgatactgatagtctttacatggataaacgagcacttgacaagtttcctaaaccaatgtttcataaaatgaatttggggggttgggatattgaacacgaaaacatcactaaattttatgcattcaatcataaaaaatattgcttgtatgataacggtattgttgtccgttgcggtggggtatcaaaatcacttattaaagaatggataaaacattcacatgatgactttgaattttttgtgaagtgttatttttcagatggaactattgtttcatcaacacgttctatcaggaacgaatataacactatctctatttataattcatcagcaaaacttgaaaaaggttttccttactttgatagctacattttacaaaatgaaaaagagctagaaaaaataaaatcacaaatacgtgatgaaatagctaaccaaaactcaaacgagcttttatatgttgaaacaccttatggttcaattggttctaatgaaataattccaaatgaagacgtgccagaaaataataacattcgtgaactaatcgaagaatataacaattttaaaagacacaatcaaaaggacttattctaaaataagccctttttctcatataataaattggttgaaaagtcacaccattcaaggttataccaagcgttattagtcttgtcatggtctatgtgatgaatggtattataattgtttggattcggtatgaacgtttcagcaactagcctatacacgtacactgttttatataaaccgttttgtgataagttaaccctttcacgtccacccttatcgtatctagtttttaagattttaccagttttcttacttttaacaaagccaccattttgaattaaatagttggtaaaaatcgggtgttctttccattcaatagatacacttttcaaatcgtttaacatctttttcactaatgtttagttcaggtaaaccaaatttttctttcaaagcaataacagtcttaatgtcagcaatatctaatcgtttgccattcaaacacgcacgatatccataattagtgtcaaaatataaacagtcgtatggatagcgcttgtcttcaatattgaaattaatctcactcatgttctttctccttaatcatttttaaaattgcagggtctacagttttaactgtttcctctttaacaacttcagtttttcctaattggtcattatcaagcatgtttttaagtttctcaggcacaggaatgaacacgctagcattttcaagaatagaaataatttccataacaagatagtatccagtgatatatgacgttaaatccgttctaagatatgcgcttaaaataatagacacaaccacaacacaccaaattgaaaccttagcaagtgccccatgcttcattttcttacttgacacgttcttttggtcgtatgctttaatgaaacccgttacaatatctaaaatattacacatcaataaatactgcattaatgtattcatatgtatatacctcctttattaaatattatatcataaaaaaagcaccttttaaaggtgcttattgtctagttaccgtctacaatatttttgataaatggataccactgttcaccaccaccatatcgagttgggagagaatcaggcacgttagcacgttcccaaaattgataaacattatcaacaccctctcgcacactttctgaattggctaaacatttctttaacgtttctgttgtcgccccctctttaaaagcgtactccatttgagtttgaagtgaataccatccctttccgttttccctaccatagttagctagtccctcacctcgtggaccagtccattggcctaaacctagtccacaatagtgacgtgcgtcactacctttataggcttcctcgtttaaaggtattgcataaagagaaagaaatgatgaccatgaaccaaacagtgtctcaactgttgggtcgtttctccagtcctcaccaccatcacgccagtaagttttcgcttcaaaagtgaacgggtcaataccgctttcaccaacaaagtttccaacaagaccagcaataccataagcattggcattaccacaattggctagacacattttagtgatatatcttaccttgtcttcatcactcatattgtcttcatcaattgatgggttagaagttggtgccccttgtggtgtatctgtagcaactgaaccttgtgatgattttagtaaactttttaaagctttttcaagctcgtttttaatctcgtcaagcgcttcatctgataaacgaactctccacaaattcatgtttctagtaagcttaacaaccttattaaacatgtacttatctgaagctgtatagacgttttgattaaacatatctttgattttattcataaattcgtttaatgttttcattaaatctattttaatatcagtgcccggtttaccagagccaggttgtggtgatgattgtgttgaacctgatggtgatgacccaccccacccgtcatcatctgaaacaacagcacctaggaagccagcccagccacctgcacatagtttacccatgtttgcatagtgtgctggtttgttaggcgttgctccgttttgttcaatggtatcataatcctctccccaatttccagctttaatagaaatgtggccagcccaactataagacccagttgtctcccaacagcagatagcaccctttggaagcgagttgtaaattctagccgagtcagcgtcatctctaggatttcctgctacagtgtgccaaccaggcgttgacaatgcaccgctagcataatcaatataaaagttttttgctgcgtcgccacgaacatatgttgttaacccagctcctaaatcacgattaagccctgagataagtgcgatacattgatacccagcgctgggaatatcaatattacccatgccagaatctgcccaagtttttgctaaatcataaaaatctttacttgttaccatattttctccttttctaagcaatcacattattttcacttaattcacttcgtgatgccaaatcagcataattgtgccacaatctcacaccaccttcaaatagtgttttgagttggtcgaaaagttctcggtcaatatcgtcaatccaataatttcccttgaactgtaaccagttagccttagacattgaattaatgttataaatttgattatcgttgttcattgcttcgtgtccaaaagcaccataatatcggcgcaagcttgttaactcttcataattaatagttgatactttcaaccaaattccatagtcaccagttttgtcaagcacactgttattatagcccccctctgaaactgtaggtggtgaaatcttccattggttcatttgtgccttttggcttcggtaatactcatattcatcagcgaacaatccagcacctttagcgggagcactagctagtccaccagcaaaaacatttgagtaaactgaaacagcgttaaatagtctgtcttttactgaattattaggatttgtaatagcgtttatacgtccagagattgtcttagaattttctagctgtcttgaataagctgtgttagctttgttaagcgtatagttattaatcaaaactggtacattatcaaacgtgttaattgtcatctggttatcacggtaaaaaccatgtcttgaatcacttgtctcacgttgtccatattcaagcgaatatatatcaatctcattaaaaccacctaaaacagatgtagcgtataacgtgttaccatctttaattttaccagtttcaaaatttaactggttaccacgatagtcagttaggtagcaattaatgacttggtctcgtaccaaataatcttcaaagtcttttaatcctaatacctctttaatttttgatttggttaaagtaaaaggaagcgttaatatattagagctattgttaccatctaacacatataagtgtgttcctttacattcaaccgtgtttaattgtgataagtcaaaaaacattgatggcactttaacaattgttttaaaattttgagttatccaaggataatcagctaacgttgttagtaagctgtctaaatctgtgcttgatacaaggtatagacttacagctgacgtaataccgtcataagtgccaccaatcgatgtttgcatttttggtttgtcttctgtaccaaaatcaccatttaatttaacacttgattgaatgatatacatgaagtcattgaactgtacaccgtcggtgttgttattggtgaatactgatgggtcaatgaacatcattgttgatgttggtaaaatgtcattacatgttcttaaatactcctctcgtcctcttaagcgttgcaaaggcaggtgctgtctgataacttcaacgttttgaagattttccagtacgtttccttgggtgtatgtcatgacaacatcaataacaaggtctaattgtgttgttctatcatttagataagtagttttaacaataaaagcatagtacgctttaccgtcccaaccatcaatgaatcgacaatagttaaaaccttgtaaactttccatatccataggaactttaacagttcctcggtcatagcgataattaaaccgataagggaacgttatggtgttgctaccagtgaaatgggtatcaaaccaattatcccgctcactatttgaattaaaatgcaatgtattttgcatgtcagtgaacattgtatttttgaataatttgaatgttgttagtttcatatttcctcctctcatatatgatattatatcacaaaaacacccgcttttaaacgggtgaatttattagattaagaaaattagaacattccctcttgtttccatactccagattgacggcgtcttgtttgaccagtgttgtcacgtccaatgtatgcaattcgttcgttgcctacatcattccaagtgtttgacgttctaagccatgttgattgttgaagtaattgaaccgctttccattcgtttgattttctaagaccgttcggttgatacattgggataacgttagtaactttaccacctacatatacagcacattcatctgaaacaactgcgcttgctagccatttgaacgctctaatttcaggtattgttatggtaccgtttggtggtacaacatagtgtcgtcttggttgtgttgctacgttaatccacccgctatcaaacggcaatccaatatcgctttttaagttccacacacgttgtaagttaccattatcgaggattttatcaatgttataacctttagcacttgttgaatgtgttgtgtaagcacgtaccgctcgaatacctccgtaatcgaatgacacagacaagtcatcatgcactgttacattagcaaggtcaagttttgcttctatacgatatttagaaactaatgtaccagcaggatagtagttgttatatacaatgtatattgtgtcatttgaggaatgtgtattatccaagtttgaaccttgaataatgtttggcctcatattgaaactaatttcacccatgaattacccccttgctccgattgttgcatcgttttcattttgaccgcttgttgtgcgaatgaatgtgttaccgtctgttgtaccagtaaacaaattaatgttaccgctagcgatatgacggtttgctgtcatgctacctgctagaacatcatcaccagaagtccaagcgcctgaacctttcaagtctgttaaaactttagtaagtgctgtttctaaaacagtgatgcgatttgataaagcggtgtcagcgttctctcgtgctgatgtttcgttgtcaatgcgtacacctaaagcggtatctgcgttttggcgtgagcttgtttcattgtcaatgcgtacacctaaagctgtatcggcgttttgtcgtgttgttgtttcgttgtcaatgcgaacgcctaaagcttcatcacctgcaatgcgtgcgttacgttcagcggtaatttttcggtctaattcttcatcagccgcgatacggtcaagaatttcttgattaatttttgctagaagtttttccaggagtggaagatagttaggtgcaaatagtccagttggtaaacattcaatagcgttctcaatattatagctttgaccgtcaaattcaactgctttttgaattgttgataaaattacttcagctttcaaaataatttcatcgtgccacgtatgacaagcattgccttcatctatccaatcattaattttagttaaatcaatgcaattagtatcttcaacttgtacgttacgacgtgcgacacggtttaacaactcaacaattgacttgataagcttattaaagtttgatagataatcataatatgatggtgcgttggtgttatagtctcggcggtcatcgtaccatggttgccagtgcccttctaaagaaaaaggatagtgtggggcgaagtatggtttgttttcttcaaagtcgatactgttaatattttccatttttttttcctttctacataatttgtgagaacaacattctgtctaggtcgttaaataggtcattatgaaaagtgtataattctctcatgcgaccgacttcaaaagtttcttgaattccaaaatcgtctgaagtcgtttcattagaattatttgacctagattttgagtgtgctgttgtgtctgcatagtcataagtttctttatcgagcgataagtcagtgttatcttgtggtaatgtgacggctaggttgttatctctagttgttacagtaccagtagaattagaagtagagttgccataagtatatacattattattaatgtattttttaccattaacataatagtcattaatcacgtctttaatgccacgaataaagctaactaaccgccagttaaacgtttcataggtttgaaacttgatggtgcgtgttaagaacttagctaggaactctgtttcaaattcttctcggactttctcgtctaaaaaatcaagaccataaaaaatagtgttacggcaaacggttttaacttcctcatcatactgaagtactttgtgggtaaattgtagttcaggattataataaacaatctggttagaacgtaaaaaatcgctgtaaatattgttataggttgtgtaaataatgtcatacaatcttgtagttgtcttcattaaatacctccatctaaaatttgtactttttcaagacttgaaagttctgaagcgatttcatctctaaagcttgctttaagcctagttttgaatttatcgttatagtgttttagcttttcatttctagcttgtaagtaaatacctgcgtttgctttttgataagctttattgctttcggcttctgattttgaaacaccgctctctttatcaacagctagagaagtaagtcctaaaatgttgtttaattcagcaatgttgttttggtattctcgttttagttcaggtaaagcactgataagtccttgaccctcgttaattgaaagaatactttcgtcagggtcaaagaattttgttgttttaataaatggtgcgccgttgtataaatcttgggtgatttgctcaacgtcctcatcgttcgcctcgcctcggataactgttgaaattttagcttgcatgtaaatgctaaagcgtgacatagcaatttcagcaatgcgttcgctgtatgtttcgatgatattaaaatcgttagttagctgaattggtttgttccaaagaacaacgaagttcccagattcatagccatcgtggtaagtgatttccttgtaatatttttctttgattttgttagagataaaaaagttaatgtcattaccagtcaaaggtctagtaccgtaggtttgaatattggaaattgtatttgattggttaacagtacctaaaatcatagttcctcttttagtttgaccaatagcaacaccgtagccttgacgtagccagacttctaattgaatagggttaattaaaacattttcatcattgataccgtcatatccaataactgccggtaaaaattcaatgtaacggtttctaaagaaattgaaaaagttatttctttgtgatacaaccctcattttaaccttttcggataaggtttgctcaatatttgaatagaagtgtgtcatttttctcctttccgtgagttgcgttaggacttttagtccttacgaaccgctctatttaaaaataaaagcagggacaagccctgcattattaggctgtttcgcctttgatgacaaccttgttatagaatggtgaaattgctttaaatgaatagtagtgaatccagtaagtgacttcatcaaattcaccgttgtagaatggttgtttcaacattccttttgtaaagcgtttgtaacgaattgctcgtgcgtctagaaccattgcaaacaagtctgattctggtttaatttcttcaaggtcttttgtaatcacgtctgaaacatcataagtgaatacatagcctttaggaataatgtcaccaacttgaacttgatagtcgccgaacaattgaagtttgttaaggatagtctgtgttacagtaatatctgctgttgtgcggtaagccccacccaagtcatcgaatgaaataatgatttttgataggtcaattccctcagcgtggaatgtgttagctaggaacgtgtttaataggtagcgtttaactttgtcagtggtgatgataatcatatctgaaagtttagtgttagtagtgaaacgacctagagcgccgcctgaagccgtagaagcttcgttgtgtttagcgctgttgttttgaaggttaaggattgcttctgaaagtgtttcaaacaattcttccatggttgtaacggtgcgagtgtcggcaacgtggtgaagaccataatcaaccagcattgcttttacttcagattcttcggcaacgttgatatcagtgattttcttgcggtatactgatacagcgtaagtgatagcgtcaccgattgttaaaaagtttaggcgagtatcgttgttgttaagtgtgaatttgagtttcttaagaataccttgtccgtaaagttttgtagccatttttggataattacgttttagcataagttcggcattcttagataggtcaagttcaattggtacactgtccaaaataacatattcttcagagtattgaccgatgaagtcaacctctttagctagccagttaaaacggtttccaagaattgactcaatgataagtgtttcgttaagttttggaaaaagatatttgttaacgtatgtttcaaattcttttccttgattgttccagttagttccaaacgtccaagcgtgacctgtttcggtgttgtgcgtgataatagaagcttgaactgcttgtgcaagtgcgtcagccattagttagtacctccaatattcataattttttgttcgtaagcgtcacgtccttgtggtgcgattgacattgattcattcaattcattggcttttgatagtttttccatgccaaaatcaccgccagagttaaatgtctttgctgttccttcgattggttccatagtgtttcctttctattctgctagaagttcttctaggtatttttgagattcaagttctgatacttcattttgaagtttttgagcgtcttcaagtgcgtgttgatatgtgataaagtgttgatacatgagtttatcaagtttagcaattttatcagtgtctttttcatcaacgattgctttagctttcaccactttaatatattcactgtaagcatttgtttgagtaatttgagcttgtgatagctgttcacgtttttgtgagagctgttcatcgatagaagtagaagtagaagttgtttgttctgccattttttaatcctttctattaagagttgagaagtttttcaatttcatctacagattcaacttcaggttctttgtcttttgttttgtcttcgtcttttggtttgtcttcatcttttgttttttcgctagaagattgttcgataacagaaagacgtgacatgatttcatctagagttgaatttaaatcgtcttttgttttgatttccaattttaaaactcctttctaaaacataaaatgtagcacaatattattagtcatactgtcatgtgttacttaattatgttttaatattgtactaatttttataatctaattataataaaaaaagaagccattgtcaataggttcattgtttggtgatagtaagtgtatacttttggcatttcatacatatgtaagtgtgtttgccttttctgttaaagatttcatatactttacaaataggacagtagtgttgtggaaattcatttgaataattcaattgtgttcacctcactaattgttatatctgaaatacctgtctttgcatttaattttgaaaataatgagtttaattgttcgctagagcattctttttcattgtaatgaccagcgtgccaatcgtaccatttgagtttgtaggttcttgtcattaatactccttatctattaaaataaaatatagtgtaataatagcactgatagcaaagaattggtaaactggtgaaattgctgttaagcatgacattgtgaaagcgagaccgaatagagttagtggtagcatagttaattacctttcttattataagaacaataaagtaacaaaaatcattagtaaaacaacgataaaagtgatgatgaatgctagtgcatatagtagaaataaagccattttattgctccttatttaattagtgaaattttgatattatcaatataacagtgtttgcctaaatttttaagggttattaaacatgtgtcatagtacctgaattgtacttcaatgttttcaacaccttcaatagtgattaagtcatatgttagtttccaagatttattgttcattttaatgcctcgtcataaattgttacattgtccgccataattagataagcaccgttttcttttagtaaattaacatataattcaaataattcttcattgcatgttataacatatgtgtgagtatcgcatggtaaattgtaagttaatgttttcattgttttgctcctttaagtgcttttttaatagtcatttctttgactagtttaatttcttgctttcttagattttcttggattgattttagatagttaattgcttcaacacttatagtcatttcaatacattcatcgatgtttttaatatcgttttgaattgttttgagtagttctttgtctga